(19) United States
(12) Patent Application Publication
(10) Pub. No.: US 2024/0254238 A1
(43) Pub. Date: Aug. 1, 2024

(54) PHARMACEUTICAL COMBINATION COMPRISING AN ANTI-CD205 ANTIBODY AND AN IMMUNE CHECKPOINT INHIBITOR

(71) Applicant: OXFORD BIOTHERAPEUTICS LTD, Abingdon (GB)

(72) Inventor: Christian ROHLFF, Abingdon (GB)

(21) Appl. No.: 18/562,921

(22) PCT Filed: May 19, 2022
(86) PCT No.: PCT/GB2022/051256

§ 371 Date: Nov. 21, 2023

(30) Foreign Application Priority Data
May 26, 2021 (GB) 2107518.9
Jun. 11, 2021 (GB) 2108387.8
Jun. 28, 2021 (GB) 2109271.3

(51) Int. Cl. C07K 16/28; A61K 47/68; A61P 35/00; A61P 37/04

(52) U.S. Cl. CPC C07K 16/2851; A61K 47/68031; A61K 47/68033; A61K 47/68037; A61K 47/6829; A61K 47/6831; A61P 35/00; A61P 37/04; C07K 16/2809; C07K 16/2818; C07K 2317/31

(57) ABSTRACT

The present invention relates to methods for increasing the anti-tumor immune response in a patient suffering from cancer, a method for the treatment or prophylaxis of cancer, and a method for enhancing the effectiveness of an inhibitor of PD1/PD-L1 interactions. Also provided are pharmaceutical combinations comprising (a) antibodies, or antigen-binding portions thereof, directed against CD205, and (b) a PD1/PD-L1 checkpoint inhibitor.

### INTRODUCTION

The present disclosure relates generally to the fields of immunology and molecular biology. More specifically, provided herein are methods for increasing the anti-tumor immune response and more specifically the T-cell mediated tumour specific response, or the number of T-cells in a patient suffering from cancer, a method for the treatment or prophylaxis of cancer, and a method for enhancing the effectiveness of an inhibitor of PD1/PD-L1 interactions. Also provided are pharmaceutical combinations comprising (a) antibodies, or antigen-binding portions thereof, directed against CD205, and (b) a PD1/PD-L1 checkpoint inhibitor.

### BACKGROUND

Dendritic cells (DCs) play a crucial role in initiating an immune responses against both, foreign and endogenous antigens. There are two types of DCs that have distinct origins and functions, myeloid dendritic cells (mDCs) and plasmacytoid dendritic cells (pDCs). Both mDCs and pDCs can efficiently induce CD4+ and CD8+ T cell responses against pathogens and both are also capable of interacting with Natural Killer (NK) cells. CD4+, CD8+ and NK cells play an important role in immune mediated anticancer response. However, pDCs as well as mDCs, can also induce tolerance to cancer by inducing Regulatory T cells (Tregs) (Ito et al. JEM, [2007]), which in turn block T cell proliferation and T cell activation.

Liu, X et al (Journal of Cancer, [2019], Vol. 10, p 6711-6715) disclose that Tregs and pDCs are the main immunosuppressive cells in the tumor microenvironment in gastric cancer. They show that patients with both, higher pDC numbers in gastric cancer tissue and peripheral blood had shorter overall survival than patients with low pDC numbers in each respective compartment. A similar negative impact on the survival of cancer patients due to the presence of DCs in the cancer tissue has been described in breast, ovarian and renal cancer.

CD205 (also known as DEC205 and Lymphocyte Antigen 75) is used by DCs as an endocytic receptor for self and foreign antigen presentation to either induce an immune response or immune tolerance. CD205 is expressed both on CD8+ mDCs and CD8+ pDCs (Shrimpton et al., 2009) CD205 distinguishes two major types of DCs. CD8+/CD205+ DCs reside in the T cell zone of the lymphoid organ and CD8−/33D1+ DCs reside in the red pulp and marginal zone (Dudziak et a. Science Vol. 315 p 107-111 [2007]. CD8+ CD205+ DCs have been reported to selectively induce immune suppressive Tregs (Yamazaki et al., 2008; Okeke and Uzonna, 2019; Simon and Bromberg, 2016; Kushwah and Hu, 2011) and the formation of Treg cells in the blood has been linked to the proportion of CD8+ CD205+ DCs within all CD11c+ DCs (Simon and Bromberg, 2016). Tregs are known to suppress tumor CD8+ or specific cytotoxic T cells (Chen et al. 2005; Li et al. 2020).

WO2009/061996 discloses isolated monoclonal antibodies which bind to human CD205 and related antibody based compositions and molecules. Also disclosed are pharmaceutical compositions comprising the antibodies, as well as therapeutic and diagnostic methods for using the antibodies.

WO2008/104806 discloses affinity reagents capable of binding to CD205 for use in the treatment or prophylaxis of cancer.

WO2015/052537 discloses specific isolated antibodies capable of binding to CD205 and their use in the treatment various cancers.

Programmed cell death 1 (PD1) and programmed cell death ligand 1 (PD-L1) are immune-checkpoint proteins whose interaction plays a major role in limiting the activity of T cells and these provide a major immune resistance mechanism by which tumor cells escape immune surveillance.

Multiple agents against PD-1/PD-L1 pathway have been developed and have been shown to be effective in the treatment of a number of cancer types.

A large number of clinical trials involving a PD1/PD-L1 checkpoint inhibitor in combination with a broad range of additional agents been undertaken in recent years. The majority of these have been combinations of PD1 with CTLA4, angiogenesis inhibitors or chemotherapy agents. The results from these trials have shown variable results (Schmidt, E. V., Semin Immunopathol; 41(1), 21-30 [2019]).

Gastric cancer is one of the most common malignant tumors of the digestive system and is one of the top 5 malignancies with regard to incidence and mortality rates. Advanced gastric cancer currently has limited treatment options with first line treatment being chemotherapy. Trastuzumab and ramucirumab have also been approved for HER-2 and VEGF positive tumors respectively where first line treatment has failed. The overall survival rate for gastric cancer worldwide is only ˜20%. Single agent immune checkpoint inhibitors have been shown to have some efficacy against gastric cancer, but to have poor efficiency (Song, X., et al. Oncology letters, 20(4), [2020]).

Endometrial cancer is the most common gynecological cancer in the US with about 50,000 women diagnosed annually. Advanced endometrial cancer is currently treated using radiation, chemo or hormone therapy. However, the development of new targeted therapies to treat refractory or recurring disease is desirable.

### BRIEF SUMMARY OF THE INVENTION

The present invention is based on the inventors surprising discovery that in cancer patients in which a specific population CD205+ immune modulatory cells are depleted, a significant increase in numbers of both CD4+ and CD8+ T-cells are seen in the peripheral blood. The inventors have also identified, that along with this increase in numbers of T-cells, a significant increase in the numbers of both CD4+ and CD8+ T-cells expressing PD1 is also seen.

The inventors have also observed that the absolute numbers of pDCs present in a patient's blood sample initially decline rapidly after treatment with a CD205-DM4 antibody drug conjugate (ADC) and are then replenished and double by day 21 after treatment. The same pattern is seen in CD205+ pDC cells. A similar same pattern is also seen in CD205+ mDC cells, which, after treatment with CD205-DM4 ADC, decline to day 8, but subsequently quadruple by day 21.

The inventors believe that the depletion of the CD205+ immune modulatory cells and the subsequent increase in CD4+ and CD8+ T-cells enhances the patient's immune response against the tumor. They further hypothesize that along with the increase in numbers of CD4+ and CD8+ T-cells, subsequent to depletion of the CD205+ immune modulatory cells the T-cells are activated. The inventors also hypothesize that the observed depletion of the CD205+ pDC population reverses immunosuppression in the CD205-DM4 ADC treated patient. This is supported by the disclosure of Liu et al, as discussed above, which suggests that pDCs are the main immunosuppressive cells in the tumor microenvironment in gastric cancer and are associated with shorter overall survival. Due to the significant increase in PD1/PD-L1 expression the enhanced immune response may be extended by administering an immune checkpoint inhibitor to prevent immunosuppression.

According to a first aspect of the present invention, there is provided a method for the treatment or prophylaxis of cancer comprising administering to a patient in need thereof a therapeutically effective amount of an antibody or antigen binding fragment thereof that modulates the population of CD205+ immune modulatory cells and a therapeutically effective amount of a composition comprising a checkpoint inhibitor.

It will be apparent to a person skilled in the art that the antibody or antigen binding fragment thereof that modulates the population of CD205+ immune modulatory cells and the composition comprising the checkpoint inhibitor can be administered simultaneously, separately or sequentially, preferably sequentially.

In one embodiment, the checkpoint inhibitor is directed towards a checkpoint protein selected from the group comprising PD1, PD-L1, PD-L2, CTLA-4, ICOS, TIGIT, CD28, TMIGD2, CD137, CD137L, CD27, OX40, OX40L, LAG3, VISTA, GITR, DNAM-1, CD96, 2B4, TIM-3, CEACAM, CRTAM, SLAMF6, Galectin-9, CD48, CD155, GITRL, CD40, CD40L, CD70, HVEM, B7-H7, B7-H3, B7-H4, ICOSL, CD80, CD86, BTLA, CD160, LIGHT, Adenosine A2a receptor, SIRP alpha, DC-SIGN, CD200R, DR3, TL1A, CD200, BTN2A1, CD47, IDO, TDO.

In one embodiment, the checkpoint inhibitor is PD1 or PD-L1, preferably PD1.

According to a second aspect of the present invention, there is provided a method for enhancing the effectiveness of an inhibitor of PD1/PD-L1 in a patient identified as being in need thereof, said method comprising administering to said patient (a) a therapeutically effective amount of an antibody or antigen binding fragment thereof that modulates the population of CD205+ immune modulatory cells and (b) a composition comprising an inhibitor of PD1/PD-L1 interactions.

It will be apparent to a person skilled in the art that the antibody or antigen binding fragment thereof that modulates the population of CD205+ immune modulatory cells and the composition comprising the inhibitor of PD1/PD-L1 interactions can be administered simultaneously, separately or sequentially, preferably sequentially.

It will be readily apparent to the skilled person that the term enhancing as used in the present context refers to increasing the level of effectiveness of the immune checkpoint inhibitor such that a higher level of cytotoxicity is seen after modulation of the population of CD205+ immune modulatory cells than prior to depletion, or to increasing the time period over which the immune checkpoint inhibitor is effective. It may be considered that the administration of an antibody or antigen binding fragment thereof that modulates the population of CD205+ immune modulatory cells acts to prime the immune system to express immune checkpoint proteins. Thus, administration of an immune checkpoint inhibitor will lead to higher and/or prolonged cytotoxicity.

According to a third aspect of the present invention, there is provided a method for increasing the anti-tumor immune response in a patient suffering from cancer comprising administering to said patient a therapeutically effective amount of an antibody or antigen binding fragment thereof that modulates the population of CD205+ immune modulatory cells.

As used in the context of the third aspect, the term ‘increasing the anti-tumor immune response’ means that a greater immune response to the cancer, as measured by an increase in the number of immune cells present in the patient, is seen subsequent to the depletion of the CD205+ immune modulatory cells than prior to depletion.

In one embodiment, the anti-tumor immune response is an immune cell mediated tumour specific response. In a preferred embodiment, the immune response is a T-cell mediated tumour specific response.

In a further embodiment, the anti-tumor immune response is a NK cell mediated tumour specific response.

According to a fourth aspect of the present invention, there is provided a method for increasing the number of T-cells in a patient suffering from cancer comprising administering to said patient an antibody or antigen binding fragment thereof which modulates the population of CD205+ immune modulatory cells.

In one embodiment the T-cells are CD8+ T-cells.

In another embodiment the T-cells are CD4+ T-cells.

In a further embodiment the T-cells are tumor specific T-cells.

According to a further aspect there is provided a method for reducing size of a tumor in a patient suffering from cancer comprising administering to said patient a therapeutically effective amount of an antibody or an antigen binding fragment thereof which modulates the population of CD205+ immune modulatory cells.

In one embodiment the tumor is a metastatic tumor. In a further embodiment, the metastatic tumor is in the lung or the liver.

For the avoidance of doubt, any of the embodiments of the invention described below refer to all earlier aspects of the invention where appropriate.

In one embodiment of the present invention, the CD205+ immune modulatory cells are CD8+. Preferably, CD205+ CD8+ immune modulatory cells are depleted.

In one embodiment, the immune modulatory cells are T-Reg cells.

In one embodiment of the present invention, the CD205+ immune modulatory cells are pDCs and/or mDCs. Preferably, the numbers of pDCs and/or mDCs are increased.

In one embodiment of the present invention, the CD205+ immune modulatory cells are CD4+. Preferably, the CD205+ CD4+ immune modulatory cells are depleted.

In one embodiment, the immune modulatory cells are T-Reg cells.

In one embodiment of the present invention, the immune modulatory cells are immune inhibitory cells.

In some embodiments, the immune modulatory cells are dendritic cells.

In one embodiment of the present invention, the patient is simultaneously, separately, sequentially or subsequently administered a cancer vaccine.

In a further embodiment of the present invention, the patient is simultaneously, separately, sequentially or subsequently administered a bispecific antibody. Preferably, the bispecific antibody is a T-cell engager (BiTE). More preferably, the bispecific antibody comprises a first binding domain which binds to CD3.

Preferably, the bispecific antibody comprises a second binding domain which binds to tumor specific antigen.

In one embodiment the patient is a patient who is refractory to, or whose cancer has progressed on, at least one chemotherapy.

In another embodiment, the patient is refractory to checkpoint modulator therapy.

In a further embodiment, the patient is ineligible for checkpoint modulator therapy.

The skilled person will understand that a patient who is ineligible for checkpoint modulator therapy is one who does meet the criteria specified for the therapeutic for a particular indication.

In one embodiment the checkpoint modulator therapy is PD1 therapy.

In a further embodiment, the patient has a cancer that is PDL1 negative or low.

The skilled person will understand that by the term low PDL1 expression it is meant a cancer having less than 50%, less than 40%, less than 30%, less than 20%, less than 10%, less than 5%, less than 4%, less than 3%, less than 2% or less than 1% PD-L1 expression.

As used herein the term PDL1 negative means a cancer having no detectable PDL1 expression using IHC.

In a further embodiment, the cancer is MSI stable.

In one embodiment, at least 20%, least 30%, least 40%, least 50%, least 60%, least 70%, least 80%, or more, of the CD8+ cells in a blood sample previously isolated from said patient are CD205+.

In another embodiment, at least 20%, least 30%, least 40%, least 50%, least 60%, least 70%, least 80%, or more, of the CD4+ cells in a blood sample previously isolated from said patient are CD205+.

In a further embodiment, least 20%, least 30%, least 40%, least 50%, least 60%, least 70%, least 80%, or more, of the pDCs and/or mDCs in a blood sample previously isolated from said patient are CD205+.

In one embodiment, the antibody or antigen binding portion thereof binds to CD205.

In one preferred embodiment, the antibody or antigen binding portion thereof which binds to CD205 for use in the methods of the present invention comprises:
a heavy chain variable region comprising:
i) a first vhCDR comprising SEQ ID NO: 5;
ii) a second vhCDR comprising SEQ ID NO: 6; and
iii) a third vhCDR comprising SEQ ID NO: 7; and
a light chain variable region comprising:
i) a first vlCDR comprising SEQ ID NO: 8;
ii) a second vlCDR comprising SEQ ID NO: 9; and
iii) a third vlCDR comprising SEQ ID NO: 10
optionally wherein any one or more of the above SEQ ID NOs independently comprise one, two, three, four or five amino acid substitutions, additions or deletions.

In one embodiment, the antibody is internalized.

In a further embodiment, the antibody or an antigen binding portion thereof for use in the methods of the present invention comprises a heavy chain variable region having at least 80%, 85%, 90%, 95% or 99% amino acid sequence identity to SEQ ID NO: 1 and a light chain variable region having at least 80%, 85%, 90%, 95% or 99% amino acid sequence identity to SEQ ID NO: 2.

Ranges intermediate to the above-recited values, e.g., heavy and light chain variable regions having at least 80-85%, 85-90%, 90-95% or 95-100% sequence identity to any of the above sequences are also intended to be encompassed by the present disclosure.

In one embodiment, the anti-CD205 antibody or an antigen-binding portion thereof for use in the methods of the present invention comprises the CDR1, CDR2, and CDR3 domains of the heavy chain variable (VH) region of the anti-CD205 antibody having the sequence shown in SEQ ID NO:1, and/or the CDR1, CDR2 and CDR3 domains of the light chain variable (VL) region of the anti-CD205 antibody having the sequence shown in SEQ ID NO:2.

In preferred embodiments, the CDRs are defined by the Kabat or Chothia systems.

In another embodiment, the antibody or an antigen-binding portion thereof for use in the methods of the present invention comprises a heavy chain variable region comprising a first vhCDR comprising SEQ ID NO:5; a second vhCDR comprising SEQ ID NO:6; and a third vhCDR comprising SEQ ID NO:7; and a light chain variable region comprising a first vlCDR comprising SEQ ID NO:8; a second vlCDR comprising SEQ ID NO:9; and a third vlCDR comprising SEQ ID NO:10.

In another embodiment, the anti-CD205 antibodies or an antigen-binding portions thereof for use in the methods of the present invention bind to human CD205 and include a heavy chain variable region comprising SEQ ID NO:1, and/or conservative sequence modifications thereof. The antibody may further include a light chain variable region comprising SEQ ID NO:2, and/or conservative sequence modifications thereof.

In another embodiment, the anti-CD205 antibody or antigen-binding portions thereof for use in the methods of the present invention comprises a heavy chain framework region comprising an amino acid sequence that is at least 80%, at least 85%, at least 90%, at least 91%, at least 92%, at least 93%, at least 94%, at least 95%, at least 96%, at least 97%, at least 98%, at least 99% identical to the framework of the heavy chain variable region of SEQ ID NO: 1 as shown in SEQ ID NOS: 12, 13, 14 and 15. In another embodiment, the anti-CD205 antibody or antigen-binding portions thereof for use in the methods of the present invention comprises a light chain framework region comprising an amino acid sequence that is at least 80%, at least 85%, at least 90%, at least 91%, at least 92%, at least 93%, at least 94%, at least 95%, at least 96%, at least 97%, at least 98%, at least 99% identical to the framework of the light chain variable region of SEQ ID NO:2 as shown in SEQ ID NOS: 16, 17, 18 and 19.

In one embodiment, the anti-CD205 antibody or antigen-binding portions thereof for use in the methods of the present invention comprises a heavy chain variable region and a light chain variable region encoded by nucleic acid sequences comprising SEQ ID NOs: 3 and 4, respectively, or nucleic acid sequences having at least 85%, 86%, 87%, 88%, 89%, 90%, 91%, 92%, 93%, 94%, 95%, 96%, 97%, 98% or 99% identity to the aforementioned nucleic acid sequences or sequences which differ from SEQ ID NOs: 3 and 4 due to degeneracy of the genetic code.

In one embodiment, the antibody or an antigen-binding portion thereof for use in the methods of the present invention further comprises a covalently-attached moiety. Preferably, said moiety is a drug. More preferably, said drug is selected from the group consisting of a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an amatoxin, CC1065 or taxol and derivatives thereof.

In a preferred embodiment, said drug is a maytansinoid selected from the group consisting of DM4 and DM1, preferably DM4.

In one embodiment, said cancer is a CD205 positive cancer.

In a preferred embodiment, the composition that modulates the population of CD205+ immune modulatory cells for use in the methods of the present invention comprises an antibody which binds to CD205 comprising:
a heavy chain variable region comprising:
i) a first vhCDR comprising SEQ ID NO: 5;
ii) a second vhCDR comprising SEQ ID NO: 6; and
iii) a third vhCDR comprising SEQ ID NO: 7; and
a light chain variable region comprising:
i) a first vlCDR comprising SEQ ID NO: 8;
ii) a second vlCDR comprising SEQ ID NO: 9; and
iii) a third vlCDR comprising SEQ ID NO: 10;
wherein said antibody is conjugated to a cytotoxic moiety comprising the maytansinoid DM4.

In one embodiment, the PD1/PD-L1 inhibitor is an antibody.

The skilled person will understand that the PD1/PD-L1 antibody can be any suitable antibody.

In preferred embodiments the anti-PD-1 antibody for use in the methods of the present invention is selected from the group comprising: Nivolumab (MDX-1 106, Opdivo; Bristol-Myers Squibb), Pembrolizumab (MK-3475, Keytruda, lambrolizumab, BMS-936558; Merck), Dostarlimab (TSR-042 Tesaro, Inc.), Cemiplimab (REGN2810, Libtayo, Regeneron Pharmaceuticals), EH12.2H7 (BioLegend, catalog no. 329902), Balstilimab (Agenus Inc).

In other preferred embodiments the anti-PD-L1 antibody for use in the methods of the present invention is selected from the group comprising: Avelumab (Bavencio; EMD Serono, Pfizer), Durvalumab (Imfinzi, AstraZeneca), BMS-936559, Atezolizumab (Tecentriq, Genentech).

In one embodiment of the present invention, the checkpoint inhibitor is administered between 7 days and 12 weeks after administration of the antibody or antigen binding portion thereof which binds to CD205, preferably between 7 days and 10 weeks, or 7 days and 8 weeks, or 7 days and 6 weeks, or 7 days and 4 weeks, or 7 and 21 days or 10 and 19 days, 12 and 16 days, 14 and 16 days, or 19 and 28 days, more preferably 20 and 25 days, most preferably 21 and 24 days.

In one embodiment of the present invention, the checkpoint inhibitor is administered 1 week, 2 weeks, 3 weeks, 4 weeks, 5 weeks or 6 weeks after administration of the antibody.

The skilled person will understand that as the number or percentage of T-cells expressing PD1 increases the immune response will be suppressed. This may be through interactions of the PD1/PD-L1 immune checkpoint. If these interactions can be prevented by, for example, administering a checkpoint inhibitor, the patient's immune response against the tumour can be sustained, leading to greater T-cell cytotoxicity against the tumour.

In one embodiment, the patient is administered at least 1 cycle, at least 2 cycles, at least 3 cycles, at least 4 cycles or at least 5 cycles of the antibody or an antigen binding fragment thereof that modulates the population of CD205+ immune modulatory cells prior to administration of the checkpoint modulator.

In another embodiment, the patient is administered 1 to 5 cycles, 2 to 4 cycles or 2 to 3 cycles of the antibody or an antigen binding fragment thereof that modulates the population of CD205+ immune modulatory cells prior to administration of the checkpoint modulator.

In some embodiments, the cancer is selected from the group consisting of pancreatic cancer, ovarian cancer, breast cancer, colorectal cancer, endometrial cancer, esophageal cancer, gastroesophageal junction cancer, skin cancer, thyroid cancer, lung cancer, kidney cancer, liver cancer, head and neck cancer, bladder cancer, gastric cancer, leukemia, preferably acute myeloid leukemia or chronic lymphocytic leukemia, myeloma, preferably multiple myeloma and lymphoma, preferably diffuse large B-cell lymphoma (DLBCL), B-Cell Lymphoma, Follicular Lymphoma, Mantle Cell Lymphoma, Lymphoma of Mucosa-Associated Lymphoid Tissue (MALT), T-Cell/Histiocyte-Rich B-Cell Lymphoma, Burkitt's Lymphoma, Lymphoplasmacytic Lymphoma, Small Lymphocytic Lymphoma, Marginal Zone Lymphoma, T Cell Lymphoma, Peripheral T-Cell Lymphoma, Anaplastic Large Cell Lymphoma and AngioImmunoblastic T-Cell Lymphoma.

Preferably, the cancer is selected from the group comprising: gastric cancer, endometrial cancer, gastroesophageal junction cancer, esophageal cancer, ovarian cancer, lung cancer, breast cancer, renal cancer and bladder cancer. Most preferably, gastric cancer.

In one embodiment, the breast cancer is triple negative breast cancer (TNBC). In another embodiment, the breast cancer is Her2-ve breast cancer.

In one embodiment, the administration of the antiCD205 antibody or antigen binding portion thereof results in an increase in the number of CD8+ T-cells in the patient leading to increased T-cell cytotoxicity against the tumour.

In preferred embodiments the patient according to any previous aspect is a human.

In some embodiments of the present invention, the antibody or antigen binding fragment thereof that modulates the population of CD205+ immune modulatory cells is an anti-CD205-DM4 ADC.

In one embodiment, the anti-CD205-DM4 ADC is administered to the patient in a dosage range from about 0.8 to 10 mg/kg, for example, 1.0 mg/kg to 8.0 mg/kg, 1.2 mg/kg to 7.5 mg/kg, 1.4 mg/kg to 7.0 mg/kg, 1.6 to 6.0 mg/kg, 1.6 to 5 mg/kg, 2.0 to 4 mg/kg, 2.5 to 3.6 mg/kg of the host body weight. For example, dosages can be 0.8 mg/kg, 1.0 mg/kg, 1.2 mg/kg, 1.4 mg/kg, 1.6 mg/kg body weight, 2.0 mg/kg body weight, 2.5 mg/kg body weight, 3.5 mg/kg body weight, 4 mg/kg body weight or 5 mg/kg body weight. Most preferably, 3.5 mg/kg. An exemplary treatment regime entails administration once every week, once every two weeks, once every three weeks, once every four weeks, once a month, once every 6 weeks, once every 3 months or once every three to 6 months.

Preferred dosage regimens for the anti-CD205-DM4 ADC for use in the methods of the invention include 2.0 mg/kg body weight, 2.5 mg/kg body weight, 3.0 mg/kg body weight, 3.5 mg/kg body weight or 5 mg/kg body weight via intravenous administration, with the antibody drug conjugate being given using one of the following dosing schedules: (i) once every 3 weeks for six dosages; (ii) once every three weeks; (iii) 2.5 mg/kg body weight once followed by 2 mg/kg body weight every three weeks.

Further preferred dosage regimens of the anti-CD205 antibody drug conjugate for use in the methods of the invention include 0.8 mg/kg body weight, 1.0 mg/kg body weight, 1.2 mg/kg body or 1.4 mg/kg body via intravenous administration, with the antibody drug conjugate being given using one of the following dosing schedules: (i) once every week; (ii) once every week for 4 dosages; (iii) once every week for 3 dosages; (iv) three times a week once every three weeks.

In one embodiment, the PD1 antibody is administered to the patient in a dosage range from 200 mg to 480 mg, for example, 200 mg, 240 mg, 400 mg or 480 mg. An exemplary treatment regime entails administration once every 2 weeks, once every three weeks, once every four weeks, once every five weeks or once every six weeks.

In another embodiment, For administration of the PD-L1 antibody, the dosage ranges from 800 mg to 1500 mg e.g. 800 mg, 1200 mg or 1500 mg. An exemplary treatment regime entails administration once every 2 weeks, once every three weeks or once every four weeks

According to a further aspect of the present invention there is provided a pharmaceutical combination comprising:
a) an anti CD205 antibody or antigen binding portion thereof, said antibody comprising:
a heavy chain variable region comprising:
i) a first vhCDR comprising SEQ ID NO: 5;
ii) a second vhCDR comprising SEQ ID NO: 6; and
iii) a third vhCDR comprising SEQ ID NO: 7; and
a light chain variable region comprising:
i) a first vlCDR comprising SEQ ID NO: 8;
ii) a second vlCDR comprising SEQ ID NO: 9; and
iii) a third vlCDR comprising SEQ ID NO: 10; and
b) a checkpoint inhibitor.

In one embodiment the pharmaceutical combination is in the form of a combined preparation for simultaneous, separate or sequential use.

In a further embodiment, the checkpoint inhibitor is a PD1/PD-L1 checkpoint inhibitor, preferably the PD1/PD-L1 checkpoint inhibitor is an antibody.

Preferably, the pharmaceutical combination is for the treatment of cancer.

In one embodiment, the PD1/PD-L1 checkpoint inhibitor is an antibody.

The skilled person will understand that the PD1/PD-L1 antibody can be any suitable antibody.

In preferred embodiments the anti-PD-1 antibody is selected from the group comprising: Nivolumab (MDX-1 106, Opdivo; Bristol-Myers Squibb), Pembrolizumab (MK-3475, Keytruda, lambrolizumab, BMS-936558; Merck), Dostarlimab (TSR-042 Tesaro, Inc.), Cemiplimab (REGN-2810, Libtayo; Regeneron), EH12.2H7 (BioLegend, catalog no. 329902).

In other preferred embodiments the anti-PD-L1 antibody is selected from the group comprising: Avelumab (Bavencio; EMD Serono, Pfizer), Durvalumab (Imfinzi, AstraZeneca), BMS-936559, Atezolizumab (Tecentriq, Genentech).

In a further embodiment, the antibody or an antigen-binding portion thereof comprises a heavy chain variable region having at least 80%, 85%, 90%, 95% or 99% amino acid sequence identity to SEQ ID NO: 1 and a light chain variable region having at least 80%, 85%, 90%, 95% or 99% amino acid sequence identity to SEQ ID NO: 2. In a preferred embodiment, the antibody or an antigen-binding portion thereof comprises a heavy chain variable region having the sequence of SEQ ID NO: 1 and the light chain variable region having the sequence of SEQ ID NO: 2.

In a further embodiment, the antibody comprises a heavy chain having at least 80%, 85%, 90%, 95% or 99% amino acid sequence identity to SEQ ID NO: 100 and a light chain having at least 80%, 85%, 90%, 95% or 99% amino acid sequence identity to SEQ ID NO: 101. In a preferred embodiment, the antibody comprises a heavy chain having the sequence of SEQ ID NO: 100 and a light chain having the sequence of SEQ ID NO: 101.

All of the antibodies disclosed herein can be full-length, for example, any of the following isotypes: IgG1, IgG2, IgG3, IgG4, IgM, IgA1, IgA2, IgAsec, IgD, and IgE. Alternatively, the antibodies can be fragments such as an antigen-binding portion or a single chain antibody (e.g., a Fab, F(ab′)₂, Fv, a single chain Fv fragment, an isolated complementarity determining region (CDR) or a combination of two or more isolated CDRs). The antibodies can be any kind of antibody, including, but not limited to, human, humanized, and chimeric antibodies.

In one embodiment, the anti-CD205 antibody or an antigen-binding portion thereof further comprises a covalently-attached moiety. Preferably, said moiety is a drug. More preferably, said drug is selected from the group consisting of a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an amatoxin, CC1065 or taxol and derivatives thereof.

In a preferred embodiment, said drug is a maytansinoid selected from the group consisting of DM4 and DM1, preferably DM4.

In a further embodiment, the pharmaceutical combination comprises at least one pharmaceutically acceptable diluent, excipient or carrier.

In a further aspect of the present invention, there is provided a composition or pharmaceutical combination of the invention for use in the treatment of cancer.

Also provided is the use of components (a) and (b) as defined above in the manufacture of a pharmaceutical combination for separate, sequential use for the treatment of cancer.

According to a further aspect of the present invention there is provided a method for selecting a patient suitable for therapy with an antibody or antigen binding fragment thereof which binds to CD205, wherein said patient is suffering from cancer, said method comprising:
identifying a patient wherein at least 20% of the CD8+ cells in a blood sample previously isolated from said patient are CD205+ and administering a therapeutically effective amount of an anti CD205 antibody or antigen binding fragment thereof to said patient.

According to a further aspect of the present invention there is provided an in vitro method for selecting patients for treatment with an antibody or antigen binding fragment thereof which binds to CD205 comprising:
a. determining the percentage of CD8+ cells in a blood sample previously isolated from said patient that are CD205+ cells; and
b. selecting the patient for treatment with the antibody or antigen binding fragment thereof which binds to CD205 if at least 20% of the CD8+ cells in the blood sample CD205+.

In one embodiment, the method for selecting a patient further comprises the step of administering to said patient a therapeutically effective amount of said antibody or antigen binding fragment thereof which binds to CD205.

According to another aspect of the present invention there is provided a method for determining the efficacy of an antibody or antigen binding fragment thereof which binds to CD205 in the treatment of cancer in a patient, said method comprising
obtaining a blood sample from said subject,
identifying whether at least 20% of the CD8+ cells in the blood sample are CD205+.

In one embodiment, the method for determining the efficacy further comprises the step of administering to said subject a therapeutically effective amount of an antibody or antigen binding fragment thereof which binds to CD205 if at least 20% of the CD8+ cells in the blood sample are CD205+.

In further embodiments, at least 30%, at least 40%, at least 50%, at least 60%, at least 70%, at least 80%, at least 90%, at least 95%, at least 96%, at least 97%, at least 98%, at least 99% of said patient's CD8+ cells are CD205+.

According to a further aspect of the present invention there is provided a method for selecting a patient suitable for therapy with an antibody or antigen binding fragment thereof which binds to CD205, wherein said patient is suffering from cancer, said method comprising:
identifying a patient wherein at least 20% of the CD4+ cells in a blood sample previously isolated from said patient are CD205+ and administering a therapeutically effective amount of an anti CD205 antibody or antigen binding fragment thereof to said patient.

According to a still further aspect of the present invention there is provided an in vitro method for selecting patients for treatment with an antibody or antigen binding fragment thereof which binds to CD205 comprising:
a. determining the percentage of CD4+ cells in a blood sample previously isolated from said patient that are CD205+ cells; and
b. selecting the patient for treatment with the antibody or antigen binding fragment thereof which binds to CD205 if at least 20% of the CD4+ cells in the blood sample CD205+.

In one embodiment, the method for selecting a patient further comprises the step of treating said patient with said antibody or antigen binding fragment thereof which binds to CD205.

According to another aspect of the present invention there is provided a method for determining the efficacy of an antibody or antigen binding fragment thereof which binds to CD205 in the treatment of cancer in a patient, said method comprising
a. obtaining a blood sample from said subject,
b. identifying whether at least 20% of the CD4+ cells in the blood sample are CD205+.

In one embodiment, the method for determining the efficacy further comprises the step of administering to said subject a therapeutically effective amount of an antibody or antigen binding fragment thereof which binds to CD205 if at least 20% of the CD4+ cells in the blood sample are CD205+.

In further embodiments, at least 30%, at least 40%, at least 50%, at least 60%, at least 70%, at least 80%, at least 90%, at least 95%, at least 96%, at least 97%, at least 98%, at least 99% of said patient's CD4+ cells are CD205+.

According to a further aspect of the present invention there is provided a method for selecting a patient suitable for therapy with an antibody or antigen binding fragment thereof which binds to CD205, wherein said patient is suffering from cancer, said method comprising: identifying a patient wherein at least 20% of the CD8+ and CD4+ cells in a blood sample previously isolated from said patient are CD205+ and administering a therapeutically effective amount of an anti CD205 antibody or antigen binding fragment thereof to said patient.

According to a still further aspect of the present invention there is provided an in vitro method for selecting patients for treatment with an antibody or antigen binding fragment thereof which binds to CD205 comprising:
a. determining the percentage of CD8+ and CD4+ cells in a blood sample isolated from said patient that are CD205+ cells; and
b. selecting the patient for treatment with the antibody or antigen binding fragment thereof which binds to CD205 if at least 20% of the CD8+ and CD4+ cells in the blood sample CD205+.

In one embodiment, the method for selecting a patient further comprises the step of treating said patient with said antibody or antigen binding fragment thereof which binds to CD205.

According to another aspect of the present invention there is provided a method for determining the efficacy of an antibody or antigen binding fragment thereof which binds to CD205 in the treatment of cancer in a patient, said method comprising
obtaining a blood sample from said subject,
identifying whether at least 20% of the CD8+ and CD4+ cells in the blood sample are CD205+.

In one embodiment, the method for determining the efficacy further comprises the step of administering to said subject a therapeutically effective amount of an antibody or antigen binding fragment thereof which binds to CD205 if at least 20% of the CD8+ and CD4+ cells in the blood sample are CD205+.

In further embodiments, at least 30%, at least 40%, at least 50%, at least 60%, at least 70%, at least 80%, at least 90%, at least 95%, at least 96%, at least 97%, at least 98%, at least 99% of said patient's CD8+ and CD4+ cells are CD205+.

According to a further aspect of the present invention there is provided a method for the treatment or prophylaxis of cancer comprising identifying a patient wherein at least 20% of the CD8+ cells in a blood sample previously isolated from said patient are CD205+ and administering to said patient a therapeutically effective amount of an antibody or antigen binding fragment thereof which binds to CD205.

Preferably, wherein at least 30%, at least 40%, at least 50%, at least 60%, at least 70%, at least 80%, at least 90%, at least 95%, at least 96%, at least 97%, at least 98%, at least 99% of said patient's CD8+ cells are CD205+.

According to a further aspect of the present invention there is provided a method for the treatment or prophylaxis of cancer comprising identifying a patient wherein at least 20% of the CD4+ cells in a blood sample previously isolated from said patient are CD205+ and administering to said patient a therapeutically effective amount of an antibody or antigen binding fragment thereof which binds to CD205.

Preferably, wherein at least 30%, at least 40%, at least 50%, at least 60%, at least 70%, at least 80%, at least 90%, at least 95%, at least 96%, at least 97%, at least 98%, at least 99% of said patient's CD4+ cells are CD205+.

According to a further aspect of the present invention there is provided a method for the treatment or prophylaxis of cancer comprising identifying a patient wherein at least 200% of the CD8+ cells and CD4+ cells in a blood sample previously isolated from said patient are CD205+ and administering to said patient a therapeutically effective amount of an antibody or antigen binding fragment thereof which binds to CD205.

Preferably, wherein at least 30%, at least 40%, at least 50%, at least 60%, at least 70%, at least 80%, at least 90%, at least 95%, at least 96%, at least 97%, at least 98%, at least 99% of said patient's CD8+ and CD4+ cells are CD205+.

According to a further aspect there is provided a treatment method comprising:
(a) calculating the percentage of CD4+ and/or CD8+ cells that are CD205+ in a blood sample previously isolated from a patient diagnosed with cancer to identify the patient as having a responder phenotype; and
(b) administering a therapeutically effective amount of an antibody or antigen binding fragment thereof which binds to CD205 to the patient having a responder phenotype.

As used herein, the term responder phenotype is defined as a patient in which at least 20%, at least 30%, at least 40%, at least 50%, at least 60%, at least 70%, at least 80%, at least 90%, at least 95%, at least 96%, at least 97%, at least 98%, at least 99% of the CD4+ and/or CD8+ cells in the blood sample previously isolated from said patient are CD205+ positive.

In one embodiment, said antibody or antigen binding fragment thereof which binds to CD205 further comprises a covalently-attached moiety. Preferably, said moiety is a drug. More preferably, said drug is selected from the group consisting of a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an amatoxin, CC1065 or taxol and derivatives thereof.

In a preferred embodiment, said drug is a maytansinoid selected from the group consisting of DM4 and DM1, preferably DM4.

In some embodiments, the method comprises the further step of subsequently administering to said patient a checkpoint inhibitor.

In certain embodiments, the checkpoint inhibitor is directed towards a checkpoint protein selected from the group comprising PD1, PD-L1, PD-L2, CTLA-4, ICOS, TIGIT, CD28, TMIGD2, CD137, CD137L, CD27, OX40, OX40L, LAG3, VISTA, GITR, DNAM-1, CD96, 2B4, TIM-3, CEACAM, CRTAM, SLAMF6, Galectin-9, CD48, CD155, GITRL, CD40, CD40L, CD70, HVEM, B7-H7, B7-H3, B7-H4, ICOSL, CD80, CD86, BTLA, CD160, LIGHT, Adenosine A2a receptor, SIRP alpha, DC-SIGN, CD200R, DR3, TL1A, CD200, BTN2A1, CD47, IDO, TDO.

Preferably, the checkpoint inhibitor is PD1 or PD-L1, more preferably PD1.

In one embodiment, the PD1/PD-L1 inhibitor is an antibody.

In some embodiments said anti-PD-1 antibody is Nivolumab (MDX-1 106, Opdivo; Bristol-Myers Squibb), Pembrolizumab (MK-3475, Keytruda, lambrolizumab, BMS-936558; Merck), Cemiplimab (REGN-2810, Libtayo; Regeneron), Dostarlimab (TSR-042, Tesaro, Inc.), EH12.2H7 (ENUM-388D4, BioLegend, catalog no. 329902), Balstilimab (Agenus Inc.).

In Further embodiments said anti-PD-L1 antibody is Avelumab (Bavencio; EMD Serono, Pfizer), Durvalumab (Imfinzi, AstraZeneca), BMS-936559, Atezolizumab (Tecentriq, Genentech).

In various embodiments the checkpoint inhibitor is administered between 7 days and 12 weeks after administration of the antibody or antigen binding portion thereof which binds to CD205, preferably between 7 days and 10 weeks, or 7 days and 8 weeks, or 7 days and 6 weeks, or 7 days and 4 weeks, or 7 and 21 days or 10 and 19 days or 12 and 16 days or 14 and 16 days or 19 and 28 days, more preferably 20 and 25 days, most preferably 21 and 24 days.

Preferably, said patient was previously not treatable with a checkpoint inhibitor.

In a further aspect of the present invention, there is provided a method for the treatment or prophylaxis of cancer comprising administering to a patient in need thereof a therapeutically effective amount of an antibody or antigen binding fragment thereof that modulates the population of CD205+ immune modulatory cells and a therapeutically effective amount of a composition comprising a cancer vaccine.

In a further aspect of the present invention there is provided a method for enhancing the effectiveness of a cancer vaccine in a patient, said method comprising administering to said patient (a) a therapeutically effective amount of an antibody or an antigen binding fragment thereof that modulates the population of CD205+ immune modulatory cells and (b) a composition comprising a cancer vaccine.

It will be apparent to a person skilled in the art that the antibody or antigen binding fragment thereof that modulates the population of CD205+ immune modulatory cells and the composition comprising the cancer vaccine can be administered simultaneously, separately or sequentially.

The skilled person will understand that as described herein, the administration of the antibody or antigen binding fragment thereof that modulates the population of CD205+ immune modulatory cells can result in an increase in numbers of both pDCs and mDCs and also an increase in the number of T-cells present in a patient's blood. They will further understand that this increase can lead to an improved response to a cancer vaccine because of the increased numbers of dendritic cells to present the antigen encoded by the cancer vaccine and the increased number of T-cells available to be activated by the presented antigens.

In a further aspect of the present invention, there is provided a method for the treatment or prophylaxis of cancer comprising administering to a patient in need thereof a therapeutically effective amount of an antibody or antigen binding fragment thereof that modulates the population of CD205+ immune modulatory cells and a therapeutically effective amount of a composition comprising a bispecific antibody.

In one embodiment the bispecific antibody is a bispecific T-cell engager (BiTE). Preferably, the bispecific antibody comprises a first binding domain which binds to CD3. More preferably, the bispecific antibody comprises a second binding domain which binds to tumor specific antigen.

It will be apparent to a person skilled in the art that the antibody or antigen binding fragment thereof that modulates the population of CD205+ immune modulatory cells and the composition comprising the bispecific antibody can be administered simultaneously, separately or sequentially.

In a further aspect there is provided a method for enhancing the effectiveness of bispecific (preferably BiTE) antibody in a patient identified as being in need thereof, said method comprising administering to said patient (a) a therapeutically effective amount of an antibody or an antigen binding fragment thereof that modulates the population of CD205+ immune modulatory cells and (b) a composition comprising a bispecific antibody.

The skilled person will understand that due to the increase in numbers of T-cells following the administration of the antibody or antigen binding fragments thereof which modulates the population of CD205+ immune modulatory cells there will be an increased number of such cells that can be activated and brought into close proximity with the target cells by the bispecific antibody (preferably BiTE), thus increasing its effectiveness in treating cancer.

The skilled person will understand that the bispecific antibody can be any suitable bispecific antibody, preferably a BiTE. For example, but not limited to, a bispecific antibody which binds to CD19 and CD3, Epcam and CD3, DLL3 and CD3 or B7H6 and CD3.

The present invention also provides a method for treating cancer in a subject said method comprising:
a. obtaining a tumor sample from said subject,
b. immunohistochemically staining said tumor sample to identify whether at least 50% of the tumor cells in the tumor sample express DCE205 at a level of at least 2+, and
c. if at least 50% of the tumor cells in the tumor sample do express DCE205 at a level of at least 2+, administering to said subject a therapeutically effective amount of an antibody or antigen binding fragment thereof which binds to CD205.

In a further aspect, the invention provides a method for treating cancer in a human patient said method comprising: identifying a patient having a tumor in which at least 50% of the tumor cells express CD205 at a level of 2+ as measured by immunohistochemistry (IHC); and administering to said patient a therapeutically effective amount of an antibody or antigen binding fragment thereof which binds to CD205.

According to a further aspect there is provided a method of selecting a patient suitable for anti CD205 antibody therapy said method comprising: identifying a patient having a tumor having at least 50% CD205 expression at a level of 2+ as measured by immunohistochemistry; and instructing a healthcare provider to administer an anti CD205 antibody or antigen binding fragment thereof to said patient.

According to a further aspect of the present invention there is provided an in vitro method for selecting cancer patients for treatment with an antibody or antigen binding fragment thereof which binds to CD205, said method comprising:
determining the expression level of CD205 in a tumor sample isolated from said patient; and
selecting the patient for treatment with the antibody or antigen binding fragment thereof which binds to CD205 if the tumor sample shows an expression level of 2+ in at least 50% of the tumor cells as determined by IHC.

In one embodiment the in vitro method further comprises the step of treating said patient with said antibody or antigen binding fragment thereof which binds to CD205.

In a further aspect of the present invention there is provided a method for determining the efficacy of an antibody or antigen binding fragment thereof which binds to CD205 in the treatment of cancer in a subject, said method comprising
obtaining a tumor sample from said subject,
immunohistochemically staining said tumor sample to identify whether at least 50% of the tumor cells in the tumor sample express DCE205 at a level of at least 2+.

In one embodiment the method further comprises the step of administering to said subject a therapeutically effective amount of an antibody or antigen binding fragment thereof which binds to CD205 if at least 50% of the tumor cells in the tumor sample do express CD205 at a level of at least 2+.

In further embodiments, at least 55%, at least 60%, at least 65%, at least 70%, at least 75%, at least 80%, at least 85%, at least 90%, at least 95% or at least 99% of the tumor cells in the tumor sample express DEC 205 at a level of at least 2+ when measured by IHC.

It will be readily apparent that IHC can be performed using any suitable protocol and any suitable antibody which binds specifically to CD205 on tumor samples. In one embodiment the antibody is an anti-CD205 antibody from Leica (Cat #: NCL-L-CD205).

In one embodiment the tumor samples are in the form of formalin fixed paraffin embedded (FFPE samples. In an alternative embodiment the samples are fresh frozen tumor samples. Also within the scope of the invention are kits comprising a pharmaceutical combination of the invention and, optionally, instructions for use. The kit can further contain a least one additional reagent or one or more additional antibodies.

Other features and advantages of the instant invention will be apparent from the following detailed description and claims.

### BRIEF DESCRIPTION OF THE DRAWINGS

FIG. 1 depicts the sequence of CD205_A1 antibody heavy chain variable region (SEQ ID NO:1). The CDR regions of the CD205_A1 antibody heavy chain are underlined.

FIG. 2 depicts the sequence of CD205_A1 antibody light chain variable region (SEQ ID NO:2). The CDR regions of CD205_A1 antibody light chain are underlined.

FIG. 3 shows in the left hand panel the change in the numbers of CD8+ T-cells in blood samples taken from a gastric cancer patient at days 1, 8, 15 and 21 after treatment with an anti-CD205-DM4 ADC at 2.5 mg/kg. The right hand panel shows the change in the numbers of CD4+ T-cells over the time course.

FIG. 4 shows in the left hand panel the change in the percentage of the total T-cell population made up of CD4+ (upper panel) and CD8+ (lower panel) T-cells during the 21 day time course after treatment with the anti-CD205-DM4 ADC at 2.5 mg/kg. The right hand panels show change in the percentage of CD4+ and CD8+ T-cells that are PD1+ over the 21 day time course.

FIG. 5 shows in the left hand panel the change in number of CD8+ T-cells present in the patient's blood that are also PD1+ over the time course. The right hand panel shows the change in the number of CD4+ T-cells present in the patient's blood that are also PD1+ over the time course.

FIG. 6 shows the change in the number of CD8+ CD205+ cells over the 21 day time course after treatment with the anti-CD205-DM4 ADC at 2.5 mg/kg.

FIG. 7 shows the change in the number of CD4+ CD205+ cells over the 21 day time course after treatment with the anti-CD205-DM4 ADC at 2.5 mg/kg.

FIG. 8 shows in the left hand panels the numbers of mDCs and pDCs in blood samples taken from a gastric cancer patient at day 1, 8, 15 and 21 after treatment with the anti-CD205-DM4 ADC at 2.5 mg/kg. The right hand panels show the change in the numbers of CD205+ mDCs and pDCs in the patient's blood over the 21 day time course.

### DETAILED DESCRIPTION OF THE INVENTION

The present disclosure relates to methods for increasing the immune response in a patient suffering from cancer and for increasing the efficacy of immune checkpoint inhibitors. Also disclosed are pharmaceutical combinations comprising an anti-CD205 antibody and an immune checkpoint inhibitor wherein the pharmaceutical combination is in the form of a combined preparation for separate or sequential use.

### CD205 Proteins

CD205 acts as an endocytic receptor to direct captured antigens from the extracellular space to a specialized antigen-processing compartment and is thought to cause a reduction in proliferation of B-lymphocytes.

According to UNIPROT, CD205 is expressed in spleen, thymus, colon and peripheral blood lymphocytes. It has been detected in myeloid and B lymphoid cell lines. Isoforms OGTA076b and OGTA076c are expressed in malignant Hodgkin's lymphoma cells called Hodgkin's and Reed-Sternberg (HRS) cells. CD205 acts as an endocytic receptor to direct captured antigens from the extracellular space to a specialized antigen-processing compartment. It causes reduced proliferation of B-lymphocytes.

Expression of CD205 has been observed in gastric pancreatic, bladder, ovarian, breast (including Her2-ve and triple negative), colorectal, kidney, endometrial, gastroesophageal junction, esophageal, skin, thyroid and lung (non-small-cell) cancers as well as Multiple Myeloma and many different subtypes of lymphomas (including DLBCL) and leukaemias.

The anti-CD205 antibody or antigen-binding portions thereof for use in the methods or combination of the present invention may, in certain cases, cross-react with the CD205 from species other than human. For example, to facilitate clinical testing, the anti-CD205 antibodies may cross react with murine or primate CD205 molecules. Alternatively, in certain embodiments, the antibodies may be completely specific for human CD205 and may not exhibit species or other types of non-human cross-reactivity.

### PD-L1 Proteins

According to UNIPROT, PD-L1 is a type I membrane protein. The protein consists of an extracellular domain between amino acids 19-238, which is comprised of one Ig-like V-type (immunoglobulin-like) domain, one Ig-like C2-type (immunoglobulin-like) domain; it further consists of one transmembrane region and one cytoplasmic region.

In some embodiments, an antibody for use in the methods or combination of the invention binds to human PD-L1.

An antibody for use in accordance with embodiments of the invention may, in certain cases, cross-react with a PD-L1 protein from a species other than a human. For example, to facilitate pre-clinical and toxicology testing, an antibody of the invention may cross react with murine or primate PD-L1 proteins. Alternatively, in certain embodiments, an antibody for use in the methods of the present invention may be specific for a human PD-L1 protein and may not exhibit species or other types of non-human cross-reactivity.

### PD1 Proteins

According to UNIPROT, PD1 is an inhibitory receptor on antigen activated T-cells that plays a critical role in induction and maintenance of immune tolerance to self. PD1 delivers inhibitory signals upon binding to ligands CD274/PDL1 and CD273/PDLG2.

The PD1-mediated inhibitory pathway is exploited by tumors to attenuate anti-tumor immunity and escape destruction by the immune system, thereby facilitating tumor survival. The interaction with CD274/PDL1 inhibits cytotoxic T lymphocytes (CTLs) effector function. The blockage of the PD1-mediated pathway results in the reversal of the exhausted T-cell phenotype and the normalization of the anti-tumor response, providing a rationale for cancer immunotherapy.

In some embodiments, an antibody for use in the methods or combination of the invention binds to human PD1.

An antibody for use in accordance with embodiments of the invention may, in certain cases, cross-react with a PD1 protein from a species other than a human. For example, to facilitate pre-clinical and toxicology testing, an antibody of the invention may cross react with murine or primate PD1 proteins. Alternatively, in certain embodiments, an antibody for use in the methods of the present invention may be specific for a human PD1 protein and may not exhibit species or other types of non-human cross-reactivity.

### Antibodies

Antibodies that find use in the methods of the present invention can take on a number of formats as described herein, including traditional antibodies as well as antibody derivatives, fragments and mimetics. In one embodiment, the invention provides antibody structures that contain a set of 6 CDRs as defined herein (including small numbers of amino acid changes as described below).

“Antibody” as used herein includes a wide variety of structures, as will be appreciated by those in the art, that in some embodiments contain at a minimum a set of 6 CDRs as defined herein; including, but not limited to traditional antibodies (including both monoclonal and polyclonal antibodies), humanized and/or chimeric antibodies, antibody fragments, engineered antibodies (e.g. with amino acid modifications as outlined below), multispecific antibodies (including bispecific antibodies), and other analogs known in the art.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

Throughout the present specification, the Kabat numbering system is generally used when referring to a residue in the variable domain (approximately, residues 1-107 of the light chain variable region and residues 1-113 of the heavy chain variable region) (e.g, Kabat et al., supra (1991)).

The CDRs contribute to the formation of the antigen-binding, or more specifically, epitope binding site of antibodies. The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein.

In some embodiments, the antibodies for use in the methods of the present invention are full length. By “full length antibody” is meant the structure that constitutes the natural biological form of an antibody, including variable and constant regions, including one or more modifications as outlined herein.

Alternatively, the antibodies for use in the methods of the present invention can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), chimeric antigen receptors (CARs) and fragments of each, respectively. Structures that rely on the use of a set of CDRs are included within the definition of “antibody”.

In one embodiment, the antibody for use in the methods of the present invention is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable region, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference).

### Chimeric and Humanized Antibodies

In some embodiments, the antibody can be a mixture from different species, e.g. a chimeric antibody and/or a humanized antibody. That is, in the present invention, the CDR sets can be used with framework and constant regions other than those specifically described by sequence herein.

In one embodiment, the antibodies for use in the methods of the present invention can be multispecific antibodies, and notably bispecific antibodies, also sometimes referred to as “diabodies”. These are antibodies that bind to two (or more) different antigens, or different epitopes on the same antigen. Diabodies can be manufactured in a variety of ways known in the art (Holliger and Winter, 1993, Current Opinion Biotechnol. 4:446-449, entirely incorporated by reference), e.g., prepared chemically or from hybrid hybridomas.

In one embodiment, the antibody for use in the methods of the present invention is a minibody. Minibodies are minimized antibody-like proteins comprising a scFv joined to a CH3 domain. Hu et al., 1996, Cancer Res. 56:3055-3061, entirely incorporated by reference. In some cases, the scFv can be joined to the Fc region, and may include some or the entire hinge region. It should be noted that minibodies are included within the definition of “antibody” despite the fact it does not have a full set of CDRs.

The antibodies disclosed for use in the methods described herein may be isolated or recombinant. “Isolated,” when used to describe the various polypeptides disclosed herein, means a polypeptide that has been identified and separated and/or recovered from a cell or cell culture from which it was expressed. Thus an isolated antibody is intended to refer to an antibody that is substantially free of other antibodies having different antigenic specificities (e.g. an isolated antibody that specifically binds to the CD205 is substantially free of antibodies that specifically bind antigens other than the CD205). Thus, an “isolated” antibody is one found in a form not normally found in nature (e.g. non-naturally occurring). An isolated antibody as defined herein may, in one embodiment, include at least one amino acid which does not occur in the “naturally” occurring antibody. This amino acid may be introduced by way of an addition or a substitution. It will be understood that the introduced amino acid may be a naturally occurring or non-naturally occurring amino acid. In some embodiments, the antibodies of the invention are recombinant proteins, isolated proteins or substantially pure proteins. An “isolated” protein is unaccompanied by at least some of the material with which it is normally associated in its natural state, for example constituting at least about 5%, or at least about 50% by weight of the total protein in a given sample. It is understood that the isolated protein may constitute from 5 to 99.9% by weight of the total protein content depending on the circumstances. For example, the protein may be made at a significantly higher concentration through the use of an inducible promoter or high expression promoter, such that the protein is made at increased concentration levels. In the case of recombinant proteins, the definition includes the production of an antibody in a wide variety of organisms and/or host cells that are known in the art in which it is not naturally produced. Ordinarily, an isolated polypeptide will be prepared by at least one purification step. An “isolated antibody,” refers to an antibody which is substantially free of other antibodies having different antigenic specificities. For instance, an isolated antibody that specifically binds to CD205 is substantially free of antibodies that specifically bind antigens other than CD205.

Isolated monoclonal antibodies, having different specificities, can be combined in a well-defined composition. Thus for example, the antibody of the invention can optionally and individually be included or excluded in a formulation, as is further discussed below.

The anti-CD205 antibodies for use in the present invention specifically bind CD205 (e.g. SEQ ID NO: 11). “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a K_{D} for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where K_{D} refers to a dissociation rate of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope. However, in the present invention, when administering ADCs of the CD205 antibodies of the invention, what is important is that the K_{D} is sufficient to allow internalization and thus cell death without significant side effects.

Also, specific binding for a particular antigen or an epitope can be exhibited, for example, by an antibody having a K_{A} or Kₐ for an antigen or epitope of at least 20-, 50-, 100-, 500-, 1000-, 5,000-, 10,000- or more times greater for the epitope relative to a control, where K_{A} or Kₐ refers to an association rate of a particular antibody-antigen interaction.

Standard assays to evaluate the binding ability of the antibodies toward CD205 can be done on the protein or cellular level and are known in the art, including for example, ELISAs, Western blots, RIAs, BIAcore® assays and flow cytometry analysis. Suitable assays are described in detail in the Examples. The binding kinetics (e.g. binding affinity) of the antibodies also can be assessed by standard assays known in the art, such as by Biacore® system analysis. To assess binding to Raji or Daudi B cell tumor cells, Raji (ATCC Deposit No. CCL-86) or Daudi (ATCC Deposit No. CCL-213) cells can be obtained from publicly available sources, such as the American Type Culture Collection, and used in standard assays, such as flow cytometric analysis.

### CD205 Antibodies

The CD205 antibodies for use in the methods of the present invention that bind to CD205 (SEQ ID NO: 11) maybe internalized when contacted with cells expressing CD205 on the cell surface These antibodies are referred to herein either as “anti-CD205” antibodies or, for ease of description, “CD205 antibodies”. Both terms are used interchangeably herein.

The CD205 antibodies for use in the methods of the present invention are internalized upon contact with cells, particularly tumor cells, which express CD205 on the surface. That is, CD205 antibodies as defined herein that also comprise drug conjugates are internalized by tumor cells, resulting in the release of the drug and subsequent cell death, allowing for treatment of cancers that exhibit CD205 expression. Internalization in this context can be measured in several ways. In one embodiment, the CD205 antibodies are contacted with cells, such as a cell line as outlined herein, using standard assays such as MAbZap. It would be clear to the skilled person that the MabZap assay is representative of the effect that would be expected to be seen with an antibody-drug conjugate (ADC). In the latter case, the ADC would be internalized, thus taking the drug into the cell. A toxic drug would have the capacity to kill the cell, i.e. to kill the targeted cancer cell. Data from MabZap assays are readily accepted by persons of skill in the art to be representative of ADC assays (Kohls, M and Lappi, D., Biotechniques, vol. 28, no. 1, 162-165).

In one embodiment, the anti-CD205 antibody for use in the methods of the present invention comprises the heavy and light chain complementarity determining regions (CDRs) or variable regions (VRs) of the particular antibody described herein (e.g., referred to herein as “CD205_A1”). Accordingly, in one embodiment, the antibody for use in the methods of the present invention comprises the CDR1, CDR2, and CDR3 domains of the heavy chain variable (VH) region of antibody CD205_A1 having the sequence shown in SEQ ID NO:1, and the CDR1, CDR2 and CDR3 domains of the light chain variable (VL) region of antibody CD205_A1 having the sequence shown in SEQ ID NO:2.

In another embodiment, the anti-CD205 antibody for use in the methods of the present invention comprises a heavy chain variable region comprising a first vhCDR comprising SEQ ID NO: 5; a second vhCDR comprising SEQ ID NO: 6; and a third vhCDR comprising SEQ ID NO:7; and a light chain variable region comprising a first vlCDR comprising SEQ ID NO:8; a second vlCDR comprising SEQ ID NO: 9; and a third vlCDR comprising SEQ ID NO: 10.

In another embodiment, the anti-CD205 antibodies for use in the methods of the present invention bind to human CD205 and include a heavy chain variable region comprising an amino acid sequence comprising SEQ ID NO:1, and conservative sequence modifications thereof. The antibody for use in the methods of the present invention may further include a light chain variable region comprising an amino acid sequence comprising SEQ ID NO:2, and conservative sequence modifications thereof.

In further embodiments, the anti-CD205 antibodies for use in the methods of the present invention bind to human CD205 and include a heavy chain variable region and a light chain variable region comprising one of the combination of sequences set out in Table 1 below:

TABLE 1

| | Heavy Chain | Light Chain |
|---|---|---|
| Antibody | Variable Region | Variable Region |
| 6H10 | SEQ ID | SEQ ID |
| | NO: 105 | NO: 106 |
| 8A3 | SEQ ID | SEQ ID |
| | NO: 113 | NO: 114 |

In a further embodiment, the anti-CD205 antibodies for use in the methods of the present invention bind to human CD205 and include a heavy chain variable region and a light chain variable region comprising the amino acid sequences set forth in SEQ ID NOs: 1 and/or 2, respectively, and conservative sequence modifications thereof. As used herein, the term conservative sequence modification refers to, for example, the substitution of an amino acid with an amino acid having similar characteristics. It is common general knowledge for one skilled in the art what such substitutions may be considered conservative. Other modifications which can be considered to be conservative sequence modifications include, for example, glycosylation.

Optionally, one or more of SEQ ID NOs: 5-10 independently comprise one, two, three, four or five conservative amino acid substitutions; optionally, one or more SEQ ID NOs: 5-10 independently comprise one or two conservative amino acid substitutions.

Preferably, the term “conservative sequence modifications” is intended to include amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function using the functional assays described herein.

In one embodiment, the anti-CD205 antibody for use in the methods of the present invention comprises a heavy chain variable region comprising SEQ ID NO:1 or a sequence that is at least 90%, at least 91%, at least 92%, at least 93%, at least 94%, at least 95%, at least 96%, at least 97%, at least 98%, at least 99% identical to SEQ ID NO: 1. In another embodiment, the anti-CD205 antibody for use in the methods of the present invention comprises a light chain variable region comprising SEQ ID NO:2 or a sequence that is at least 90%, at least 91%, at least 92%, at least 93%, at least 94%, at least 95%, at least 96%, at least 97%, at least 98%, at least 99% identical to SEQ ID NO: 2. In another embodiment, the anti-CD205 antibody for use in the methods of the present invention comprises a heavy chain framework region comprising an amino acid sequence that is at least 90%, at least 91%, at least 92%, at least 93%, at least 94%, at least 95%, at least 96%, at least 97%, at least 98%, at least 99% identical to the framework of the heavy chain variable region of SEQ ID NO: 1 comprising SEQ ID NOs: 12, 13, 14 and 15. In another embodiment, the anti-CD205 antibody for use in the methods of the present invention comprises a light chain framework region comprising an amino acid sequence that is at least 90%, at least 91%, at least 92%, at least 93%, at least 94%, at least 95%, at least 96%, at least 97%, at least 98%, at least 99% identical to the framework of the light chain variable region of SEQ ID NO:2 comprising SEQ ID NOs: 16, 17, 18 and 19.

In one embodiment, the anti-CD205 antibody for use in the methods of the present invention is referred to herein as “CD205_A1 antibody” comprising the following CDRs, as well as variants containing a limited number of amino acid variants:

TABLE 2

| A1 | SEQ ID NOs |
|---|---|
| variable heavy | 5 |
| CDR1 | |
| variable heavy | 6 |
| CDR2 | |
| variable heavy | 7 |
| CDR3 | |
| variable light | 8 |
| CDR1 | |
| variable light | 9 |
| CDR2 | |
| variable light | 10 |
| CDR3 | |

Disclosed herein are also variable heavy and light chains that comprise the CDR sets of the invention, as well as full length heavy and light chains (e.g. comprising constant regions as well). As will be appreciated by those in the art, the CDR sets of the anti-CD205 antibody can be incorporated into murine, humanized or human constant regions (including framework regions). Accordingly, the present disclosure provides variable heavy and light chains that are at least about 90%-99% identical to the SEQ IDs disclosed herein, with 90, 91, 92, 93, 94, 95, 96, 97, 98 and 99% all finding use in the present invention.

In one embodiment, the antibody for use in the methods of the present invention specifically binds to human CD205 comprising SEQ ID NO: 11. Preferably, the anti-CD205 antibody for use in the methods of the present invention binds to human CD205 with high affinity.

### Antibody Modifications

The present invention further provides variant antibodies for use in the methods of the present invention, sometimes referred to as “antibody derivatives” or “antibody analogs” as well. That is, there are a number of modifications that can be made to the antibodies of the invention, including, but not limited to, amino acid modifications in the CDRs (affinity maturation), amino acid modifications in the framework regions, amino acid modifications in the Fc region, glycosylation variants, covalent modifications of other types (e.g. for attachment of drug conjugates, etc.).

By “variant” herein is meant a polypeptide sequence that differs from that of a parent polypeptide by virtue of at least one amino acid modification. In this case, the parent polypeptide is either the full length variable heavy or light chains, listed in SEQ ID Nos: 1 or 2, respectively or the CDR regions or the framework regions of the heavy and light chains listed in SEQ ID NOs 5-10 and 12-19. Amino acid modifications can include substitutions, insertions and deletions, with the former being preferred in many cases. It will be understood that an amino acid substitution may be a conservative or non-conservative substitution with conservative substitutions being preferred. Further said substitution may be a substitution with either a naturally or non-naturally occurring amino acid.

By “amino acid substitution” or “substitution” herein is meant the replacement of an amino acid at a particular position in a parent polypeptide sequence with another amino acid which may be a natural or non-naturally occurring amino acid. For example, the substitution S100A refers to a variant polypeptide in which the serine at position 100 is replaced with alanine. By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

By “parent polypeptide”, “parent protein”, “precursor polypeptide”, or “precursor protein” as used herein is meant an unmodified polypeptide that is subsequently modified to generate a variant. In general, the parent polypeptides herein are LY75_A1. Accordingly, by “parent antibody” as used herein is meant an antibody that is modified to generate a variant antibody.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified.

By “variant Fc region” herein is meant an Fc sequence that differs from that of a wild-type Fc sequence by virtue of at least one amino acid modification. Fc variant may refer to the Fc polypeptide itself, compositions comprising the Fc variant polypeptide, or the amino acid sequence.

In some cases, amino acid modifications in the CDRs are referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of LY75_A1. In addition, as outlined below, amino acid modifications can also independently and optionally be made in any region outside the CDRs, including framework and constant regions as described herein.

In some embodiments, the anti-LY75 antibodies are composed of a variant Fc domain. As is known in the art, the Fc region of an antibody interacts with a number of Fc receptors and ligands, imparting an array of important functional capabilities referred to as effector functions. In addition, modifications at cysteines are particularly useful in antibody-drug conjugate (ADC) applications, further described below. In some embodiments, the constant region of the antibodies can be engineered to contain one or more cysteines that are particularly “thiol reactive”, so as to allow more specific and controlled placement of the drug moiety. See for example U.S. Pat. No. 7,521,541, incorporated by reference in its entirety herein.

### Antibody-Drug Conjugates

In some embodiments, the anti-CD205 antibodies or antigen binding portions thereof for use in the methods of the present invention disclosed herein are conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc. An overview of this technology is provided in Ducry et al., Bioconjugate Chem., 21:5-13 (2010), Carter et al., Cancer J. 14(3): 154 (2008) and Senter, Current Opin. Chem. Biol. 13:235-244 (2009), all of which are hereby incorporated by reference in their entirety.

Thus, the invention provides pharmaceutical combinations comprising, inter alia, anti-CD205 antibodies conjugated to drugs. Generally, conjugation is done by covalent attachment to the antibody, as further described below, and generally relies on a linker, often a peptide linkage (which, as described below, may be designed to be sensitive to cleavage by proteases at the target site or not). In addition, as described above, linkage of the linker-drug unit (LU-D) can be done by attachment to cysteines within the antibody. As will be appreciated by those in the art, the number of drug moieties per antibody can change, depending on the conditions of the reaction, and can vary from 1:1 to 10:1 drug:antibody. As will be appreciated by those in the art, the actual number is an average.

Thus the anti-CD205 antibodies may be conjugated to drugs. As described below, the drug of the ADC can be any number of agents, including but not limited to cytotoxic agents such as chemotherapeutic agents, growth inhibitory agents, toxins (for example, an enzymatically active toxin of bacterial, fungal, plant, or animal origin, or fragments thereof), or a radioactive isotope (that is, a radioconjugate) are provided. In other embodiments, the invention further provides methods of using the ADCs.

Drugs for use in the present invention include cytotoxic drugs, particularly those which are used for cancer therapy. Such drugs include, in general, DNA damaging agents, anti-metabolites, natural products and their analogs. Exemplary classes of cytotoxic agents include the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, dolastatins, maytansinoids, differentiation inducers, and taxols.

Members of these classes include, for example, taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoids (including DM1), monomethylauristatin E (MMAE), monomethylauristatin F (MMAF), and maytansinoids (DM4) and their analogues.

Toxins may be used as antibody-toxin conjugates and include bacterial toxins such as diphtheria toxin, plant toxins such as ricin, small molecule toxins such as geldanamycin (Mandler et al (2000) J. Nat. Cancer Inst. 92(19): 1573-1581; Mandler et al (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342), hemiasterlins (WO2004/026293; Zask et al., (2004) J. Med. Chem, 47: 4774-4786). Toxins may exert their cytotoxic and cytostatic effects by mechanisms including tubulin binding, DNA binding, or topoisomerase inhibition.

Conjugates of an anti-CD205 antibody and one or more small molecule toxins, such as a maytansinoids, dolastatins, auristatins, a trichothecene, calicheamicin, and CC1065, and the derivatives of these toxins that have toxin activity, may also be used.

Preferably, the anti-CD205 antibody is conjugated to DM1 or DM4, most preferably to DM4.

### Linker Units

Typically, the antibody-drug conjugate compounds comprise a Linker unit between the drug unit and the antibody unit. In some embodiments, the linker is cleavable under intracellular or extracellular conditions, such that cleavage of the linker releases the drug unit from the antibody in the appropriate environment. For example, solid tumors that secrete certain proteases may serve as the target of the cleavable linker; in other embodiments, it is the intracellular proteases that are utilized. In yet other embodiments, the linker unit is not cleavable and the drug is released, for example, by antibody degradation in lysosomes.

In some embodiments, the linker is cleavable by a cleaving agent that is present in the intracellular environment (for example, within a lysosome or endosome or caveolea). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long or more.

Cleaving agents can include, without limitation, cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Peptidyl linkers that are cleavable by enzymes that are present in CD205-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other examples of such linkers are described, e.g., in U.S. Pat. No. 6,214,345, incorporated herein by reference in its entirety and for all purposes.

In some embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker).

In other embodiments, the cleavable linker is pH-sensitive, that is, sensitive to hydrolysis at certain pH values. Typically, the pH-sensitive linker hydrolyzable under acidic conditions.

In yet other embodiments, the linker is cleavable under reducing conditions (for example, a disulfide linker).

In other embodiments, the linker is a malonate linker (Johnson et al., 1995, Anticancer Res. 15:1387-93), a maleimidobenzoyl linker (Lau et al., 1995, Bioorg-Med-Chem. 3(10): 1299-1304), or a 3′-N-amide analog (Lau et al., 1995, Bioorg-Med-Chem. 3(10): 1305-12).

In yet other embodiments, the linker unit is not cleavable and the drug is released by antibody degradation. (See U.S. Publication No. 2005/0238649 incorporated by reference herein in its entirety and for all purposes).

In many embodiments, the linker is self-immolative. As used herein, the term “self-immolative Spacer” refers to a bifunctional chemical moiety that is capable of covalently linking together two spaced chemical moieties into a stable tripartite molecule. It will spontaneously separate from the second chemical moiety if its bond to the first moiety is cleaved. See for example, WO 2007/059404A2, WO06/110476A2, WO05/112919A2, WO2010/062171, WO09/017394, WO07/089149, WO 07/018431, WO04/043493 and WO02/083180.

Often the linker is not substantially sensitive to the extracellular environment. As used herein, “not substantially sensitive to the extracellular environment,” in the context of a linker, means that no more than about 20%, 15%, 10%, 5%, 3%, or no more than about 1% of the linkers, in a sample of antibody-drug conjugate compound, are cleaved when the antibody-drug conjugate compound presents in an extracellular environment (for example, in plasma).

In other, non-mutually exclusive embodiments, the linker promotes cellular internalization. In certain embodiments, the linker promotes cellular internalization when conjugated to the therapeutic agent (that is, in the milieu of the linker-therapeutic agent moiety of the antibody-drug conjugate compound as described herein). In yet other embodiments, the linker promotes cellular internalization when conjugated to both the auristatin compound and the anti-CD205 antibodies of the invention.

A variety of exemplary linkers that can be used with the present compositions and methods are described in WO 2004/010957, U.S. Publication No. 2006/0074008, U.S. Publication No. 20050238649, and U.S. Publication No. 2006/0024317 (each of which is incorporated by reference herein in its entirety and for all purposes).

Preferably, the linker is SPDB (N-succinimidyl-3-(2-pyridyldithio)butyrate).

### Pharmaceutical Compositions

### Combinations

The pharmaceutical combination of the invention is in the form of a combined preparation for separate or sequential use. Similarly, in the methods of the invention, components (a) and (b) of the pharmaceutical combination may be administered to a patient separately or sequentially.

The term “pharmaceutical combination” as used herein refers to a pharmaceutical product comprising at least two active ingredients either in a single formulation or as individual components.

The term “combined preparation” as used herein means a preparation comprising both components a) and b) either as individual components or in a single formulation.

Where the administration is sequential, the delay in administering the second component should be such that the benefit of the effect arising from use of the combination is maximized. Therefore, in one embodiment sequential treatment involves administration of each component of the combination within a period of 84 days. In another embodiment this period is 77 days. In another embodiment this period is 70 days. In another embodiment this period is 63 days. In another embodiment this period is 56 days. In another embodiment this period is 49 days. In another embodiment this period is 42 days. In another embodiment this period is 35 days. In another embodiment this period is 28 days. In another embodiment this period is 24 days. In another embodiment this period is 21 days. In another embodiment this period is 18 days. In another embodiment this period is 15 days. In another embodiment this period is 13 days. In another embodiment this period is 11 days. In another embodiment this period is within 9 days. In another embodiment this period is within 7 days. In another embodiment this period is within 5 days. In another embodiment this period is within 3 days. In another embodiment this period is within 1 day. In a preferred embodiment, the sequential treatment involves administration of each component of the combination within a period of 14-16 days.

Components (a) should be administered first and then component (b).

The ratio of the total amounts of component (a) to component (b) to be administered in the combined preparation can be varied, e.g. in order to cope with the needs of a patient sub-population to be treated or the needs of the single patient which different needs can be due to age, sex, body weight, etc. of the patients.

Components (a) and (b), whether present in a single composition or in separate compositions, may independently be formulated with one or more pharmaceutically-acceptable carriers. The pharmaceutical combinations of the invention may also include at least one other anti-tumor agent, or an anti-inflammatory or immunosuppressant agent. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion). Depending on the route of administration, the active compound, i.e. antibody, immunoconjugate, or bispecific molecule, may be coated in a material to protect the compound from the action of acids and other natural conditions that may inactivate the compound.

Examples of suitable aqueous and non-aqueous carriers that may be employed in the pharmaceutical combinations of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These combinations or parts thereof may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of 100 percent, this amount will range from about 0.01 percent to about 99 percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the anti-CD205-DM4 ADC the dosage ranges from about 0.8 to 10 mg/kg, for example, 1.0 mg/kg to 8.0 mg/kg, 1.2 mg/kg to 7.5 mg/kg, 1.4 mg/kg to 7.0 mg/kg, 1.6 to 6.0 mg/kg, 1.6 to 5 mg/kg, 2.0 to 4 mg/kg, 2.5 to 3.6 mg/kg of the host body weight. For example, dosages can be 0.8 mg/kg, 1.0 mg/kg, 1.2 mg/kg, 1.4 mg/kg, 1.6 mg/kg body weight, 2.0 mg/kg body weight, 2.5 mg/kg body weight, 3.5 mg/kg body weight, 4 mg/kg body weight or 5 mg/kg body weight. An exemplary treatment regime entails administration once every week, once every two weeks, once every three weeks, once every four weeks, once a month, once every 6 weeks, once every 3 months or once every three to 6 months.

Preferred dosage regimens of the anti-CD205-DM4 ADC for use in the methods of the invention include 2.0 mg/kg body weight, 2.5 mg/kg body weight, 3.0 mg/kg body or 3.5 mg/kg body weight via intravenous administration, with the antibody drug conjugate being given using one of the following dosing schedules: (i) every 3 weeks for six dosages; (ii) every three weeks; (iii) 2.5 mg/kg body weight once followed by 2 mg/kg body weight every three weeks.

Further preferred dosage regimens of the anti-CD205 antibody drug conjugate for use in the methods of the invention include 0.8 mg/kg body weight, 1.0 mg/kg body weight, 1.2 mg/kg body or 1.4 mg/kg body via intravenous administration, with the antibody drug conjugate being given using one of the following dosing schedules: (i) once every week; (ii) once every week for 4 dosages; (iii) once every week for 3 dosages; (iv) three times a week once every three weeks.

For administration of the PD1 antibody, the dosage ranges from 200 mg to 480 mg, e.g. 200 mg, 240 mg, 400 mg, or 480 mg. An exemplary treatment regime entails administration once every 2 weeks, once every three weeks, once every four weeks, once every five weeks or once every six weeks.

For administration of the PD-L1 antibody, the dosage ranges from 800 mg to 1500 mg e.g. 800 mg, 1200 mg or 1500 mg. An exemplary treatment regime entails administration once every 2 weeks, once every three weeks or once every four weeks

In some methods, two or more monoclonal antibodies with different binding specificities are administered simultaneously, in which case the dosage of each antibody administered falls within the ranges indicated.

Actual dosage levels of the active ingredients in the pharmaceutical combinations of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-CD205 antibody or a combination of the invention preferably results in a decrease in severity of disease symptoms, an increase in frequency and duration of disease symptom-free periods, or a prevention of impairment or disability due to the disease affliction. For example, for the treatment of the CD205 or PD1/PD-L1 mediated tumors, a “therapeutically effective dosage” preferably inhibits cell growth or tumor growth by at least about 20%, at least about 30%, more preferably by at least about 40%, at least about 50% even more preferably by at least about 60%, at least about 70% and still more preferably by at least about 80% or at least about 90%, relative to untreated subjects. The ability of a compound to inhibit tumor growth can be evaluated in an animal model system predictive of efficacy in human tumors. Alternatively, this property of a composition can be evaluated by examining the ability of the compound to inhibit cell growth, such inhibition can be measured in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A pharmaceutical combination of the present invention can be administered via one or more routes of administration using one or more of a variety of methods known in the art. Components (a) and (b) may be administered by the same route or by different routes. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, antibody can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art [see, e.g. Sustained and Controlled Release Drug Delivery Systems (1978) J. R. Robinson, ed., Marcel Dekker, Inc., N.Y].

Therapeutic compositions can be administered with medical devices known in the art. For example, in a preferred embodiment, the antibody or antibodies can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-CD205 and/or anti-PD1/PD-L1 antibodies can be formulated to ensure proper distribution in vivo. For example, the blood-brain barrier (BBB) excludes many highly hydrophilic compounds. To ensure that the therapeutic compounds cross the BBB (if desired), they can be formulated, for example, in liposomes. For methods of manufacturing liposomes, see, e.g. U.S. Pat. Nos. 4,522,811; 5,374,548; and 5,399,331. The liposomes may comprise one or more moieties which are selectively transported into specific cells or organs, thus enhance targeted drug delivery [see, e.g. V. V. Ranade (1989) J. Clin. Pharmacol. 29:685]. Exemplary targeting moieties include folate or biotin (see, e.g. U.S. Pat. No. 5,416,016); mannosides [Umezawa et al. (1988) Biochem. Biophys. Res. Commun. 153: 1038]; antibodies [P. G. Bloeman et al. (1995) FEBS Lett. 357:140; M. Owais et al. (1995) Antimicrob. Agents Chemother. 39:180]; surfactant protein A receptor [Briscoe et al. (1995) Am. J. Physiol. 1233:134]; p 120 [Schreier et al. (1994) J. Biol. Chem. 269:9090]; see also K. Keinanen; M. L. Laukkanen (1994) FEBS Lett. 346:123; J. J. Killion; I. J. Fidler (1994) Immunomethods 4:273.

### Uses and Methods

As used herein, the term “subject” is intended to include human and non-human animals. Non-human animals include all vertebrates, e.g. mammals and non-mammals, such as non-human primates, sheep, dogs, cats, cows, horses, chickens, amphibians, and reptiles. Preferred subjects include human patients having disorders mediated by CD205 activity and/or PD1/PD-L1 activity. Suitable routes of administering the antibody compositions (e.g. monoclonal antibodies, and immunoconjugates) in vivo and in vitro are well known in the art and can be selected by those of ordinary skill. For example, the antibody compositions can be administered by injection (e.g. intravenous or subcutaneous). Suitable dosages of the molecules used will depend on the age and weight of the subject and the concentration and/or formulation of the antibody composition.

As previously described, the anti-CD205 and/or anti-PD1/PD-L1 antibodies can be co-administered with one or other more therapeutic agents, e.g. a cytotoxic agent, a radiotoxic agent or an immunosuppressive agent. The antibody can be linked to the agent (as an immunocomplex) or can be administered separate from the agent. In the latter case (separate administration), the antibody can be administered before, after or concurrently with the agent or can be co-administered with other known therapies, e.g. an anti-cancer therapy, e.g. radiation. Such therapeutic agents include, among others, anti-neoplastic agents. Other agents suitable for co-administration with the antibodies of the invention include other agents used for the treatment of cancers, e.g. gastric cancer, endometrial cancer, colorectal cancer, prostate cancer, breast cancer, ovarian cancer or lung cancer. Co-administration of the anti-CD205 antibodies or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

The pharmaceutical combinations of the invention can also be administered together with serum and/or complement. These compositions can be advantageous when the complement is located in close proximity to the antibodies. Alternatively, the antibodies, and the complement or serum can be administered separately.

Also within the scope of the present invention are kits comprising components (a) and (b), together with instructions for use. The kit can further contain one or more additional reagents, such as an immunosuppressive reagent, a cytotoxic agent or a radiotoxic agent, or one or more additional antibodies (e.g. an antibody having a complementary activity which binds to an epitope in the CD205 antigen distinct from the first antibody).

Accordingly, patients treated with pharmaceutical combinations of the invention can be additionally administered (prior to, simultaneously with, or following administration of an antibody disclosed herein) another therapeutic agent, such as a cytotoxic or radiotoxic agent, which enhances or augments the therapeutic effect of the antibodies.

All references cited in this specification, including without limitation all papers, publications, patents, patent applications, presentations, texts, reports, manuscripts, brochures, books, internet postings, journal articles, periodicals, product fact sheets, and the like, one hereby incorporated by reference into this specification in their entireties. The discussion of the references herein is intended to merely summarize the assertions made by their authors and no admission is made that any reference constitutes prior art and Applicants' reserve the right to challenge the accuracy and pertinence of the cited references.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the dependent claims.

The present invention is further illustrated by the following examples which should not be construed as further limiting.

### EXAMPLES

### Example 1: Generation of Human Monoclonal Antibodies Against CD205-Antigen

Following standard procedures, mice (xenomouse IgG1) were immunized with CHO cells transfected with full length CD205.

The specificity of antibodies raised against the CD205 was tested by flow cytometry on HEK293 cells transfected with CD205 and subsequently on CD205-expressing HT29 cells. To test the ability of the antibodies to bind to the cell surface CD205 protein, the antibodies were incubated with the CD205-expressing cells. Cells were washed in FACS buffer (DPBS, 2% FBS), centrifuged and resuspended in 100 μl of the diluted primary CD205 antibody (also diluted in FACS buffer). The antibody-cell line complex was incubated on ice for 60 min and then washed twice with FACS buffer as described above. The cell-antibody pellet was resuspended in 100 μl of the diluted secondary antibody (also diluted in FACS buffer) and incubated on ice for 60 min on ice. The pellet was washed as before and resuspended in 200 μl FACS buffer. The samples were loaded onto the BD FACScanto II flow cytometer and the data analyzed using the BD FACSdiva software (results not shown).

### Example 2: Structural Characterization of Monoclonal Antibodies to CD205

The cDNA sequences encoding the heavy and light chain variable regions of the CD205_A1 monoclonal antibody were obtained using standard PCR techniques and were sequenced using standard DNA sequencing techniques.

The antibody sequences may be mutagenized to revert back to germline residues at one or more residues.

The nucleotide and amino acid sequences of the heavy chain variable region of CD205_A1 are shown in SEQ ID NO: 3 and 1, respectively.

The nucleotide and amino acid sequences of the light chain variable region of CD205_A1 are shown in SEQ ID NO: 4 and 2, respectively.

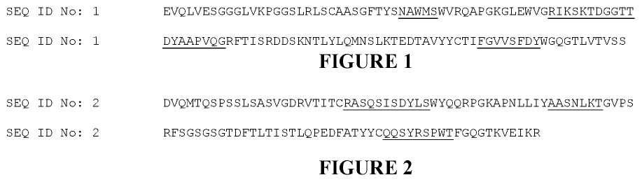

Further analysis of the CD205_A1 V_{H} sequence using the Kabat system of CDR region determination led to the delineation of the heavy chain CDR1, CDR2 and CDR3 regions as shown in SEQ ID NOs: 5, 6 and 7, respectively. The sequence of the CD205_A1 CDR1, CDR2 and CDR3 V_{H} sequences are shown in FIG. 1.

Further analysis of the CD205_A1 V_{K} sequence using the Kabat system of CDR region determination led to the delineation of the light chain CDR1, CDR2 and CDR3 regions as shown in SEQ ID NOs:8, 9 and 10, respectively. The sequences of the CD205_A1 CDR1, CDR2 and CDR3 V_{K} sequences are shown in FIG. 2.

### Example 3: Efficacy of Different DM4-Conjugated Anti-LY75 Monoclonal Antibodies in Raji and THP1 Cells

THP-1 and Raji cells were prepared to a seeding density of 3,000 cells/well (1.5×10⁵ cells/mL) and added to the assay plates (20 L/well).

THP-1 cells were prepared in RPMI GLUTAMAX Growth (2ME) Raji cells were prepared in RPMI 1640 ATCC Growth AB-Free (10%).

Each conjugated antibody was prepared in triplicate to a starting concentration at 2× the final concentration and diluted to the final concentration in RPMI 1640 ATCC Growth AB-Free (10%). Antibodies were transferred to the required assay plate and incubated for 96 hours.

Following assay incubation, Cell-Titer Glo was added to each plate and read using a plate reader set on luminescence with 0.2 sec integration.

Raw data was converted to % Specific Death (Data not shown) using the negative control (target cells only) and the EC₅₀ calculated. The EC₅₀ of the antibodies against the two cell lines is shown in Table 3. As can be seen from Table 3 antibody conjugate CD205_A1 showed a lower EC₅₀ than the other 2 antibodies tested, however all 3 conjugates showed cytotoxicity against both Raji and THP1 cells.

TABLE 3

| Cell Line | Antibody | EC₅₀ (pM) | Cytotoxicity |
|---|---|---|---|
| Raji | 16A5 (CD205_A1) | 727.6 | Yes |
| Raji | 8A3 | 1008.0 | Yes |
| Raji | 16H10 | 2168.0 | Yes |
| THP1 | 16A5 (CD205_A1) | 37.5 | Yes |
| THP1 | 8A3 | 98.8 | Yes |
| THP1 | 16H10 | 46.9 | Yes |

### Example 4: Toxicity of DM1-Conjugated and DM4-Conjugated Anti-CD205 Monoclonal Antibodies in Cynomolgus Monkeys

Six male monkeys were assigned to the study with 2 monkeys/group. Either vehicle (PBS), CD205_DM4 (cleavable) or CD205_DM1 (non-cleavable) was administered twice (on Day 1 and Day 29) by a 15-minute intravenous infusion at 0 mg/kg/dose (PBS, vehicle), 5 mg/kg/dose (CD205_DM4, cleavable) or 10 mg/kg/dose (CD205_DM1, non-cleavable). Blood samples were collected for toxicokinetic evaluations prior to dose initiation (Day 1), and 1, 2, 3, 7, 14, 21 and 28 days post each dose. Blood samples for clinical pathology analyses were collected prior to dose initiation (Day 1), and 1, 3, 7, 14, 21 and 28 days post each dose (28 days post the 1st dose was also served as the pre-dose time point for the 2nd dose). All study animals were euthanized and necropsied following the final blood collection on Day 57. The plasma separated from each blood draw was isolated, frozen and shipped to Oxford BioTherapeutics, Inc. to be analyzed for ADC concentration by ELISA.

Treatment-related clinical pathology findings included a mild regenerative anemia and transient decreases in the blood leukocyte profile most notably in neutrophils counts. Anemia was observed in both animals treated with 5 mg/kg CD205_DM4 and in one of the two animals treated with 10 mg/kg CD205_DM1. Severe neutropenia with a nadir at one-week post dose and a rapid recovery in counts was observed in all animals; the nadir in absolute neutrophil count was lower in CD205_DM4 treated animals. There were no test article-related effects on the APTT and PT coagulation parameters. Serum chemistry changes included transient increases in AST, CK, LDH (in 1 of 2 animals in each treatment group) and globulin following administration of 5 mg/kg CD205_DM4 and 10 mg/kg CD205_DM1. In addition, a transient increase in the liver specific enzyme ALT was observed only in the CD205_DM4 treated animals. The short duration of and/or the magnitude of the increases in serum chemistry parameters suggest they were not adverse. There were no test-article related urinalysis findings. Upon examination at necropsy following a 4-week recovery period there were no treatment related gross pathology findings or changes in absolute and relative organ weights. Histopathology findings only in the thyroid gland (an alteration in the colloid morphology in follicles) and kidney (dilated tubules in the outer cortex), were graded as minimal severity; not associated with changes in other study parameters; and, not adverse and of minimal toxicological significance. Conclusion: Repeated dose treatment with two doses of 5 mg/kg CD205_DM4 or 10 mg/kg CD205_DM1 was well tolerated in cynomologus monkeys. All treatment-related toxicity findings were reversible following a 4-week recovery period.

### Example 5: CD205 Immunohistochemistry Protocol

CD205 target expression level is assessed in formalin-fixed paraffin-embedded (FFPE) human tumors using immunohistochemical (IHC) staining assay. FFPE tissues were sectioned on a rotary microtome at 4-6 micron thickness and mounted on positively charged glass slides. The mounted sections were allowed to air dry on the slide at room temperature overnight, or at 37ºC for 30 minutes followed by baking at 60° C. for 30 minutes. The slides were deparaffinized in three changes of xylene for 5 minutes each and rehydrated in graded ethanols starting with three changes of 100% ethanol, followed by 1 change in 95% ethanol, 1 change in 80% ethanol and two rinses in deionized water all for 3 minutes in each solution exchange. After the deparaffinization and rehydration process, the slides underwent heat-induced epitope retrieval (HIER), in a Biocare Decloaker NxGen pressure cooker in Diva Decloaker solution (DDV2004). The slides were exposed to a temperature of 110° C. for 15 minutes and allowed to cool for an additional 10 minutes in the unit before removing. After removal from the pressure cooker, the slides were equilibrated to room temperature by gradual replacement of the hot Diva retrieval solution with deionized or distilled water. The slides were rinsed in Tris-Buffered Saline (TBS) (TWB945) and loaded into the staining racks of the intelliPATH automated staining instrument (IPS0001US). The slides were incubated for 5 minutes in 300 ul of Peroxidazed 1 (PX968) to block endogenous peroxidases. The Peroxidazed 1 was then removed and the slides were incubated for 10 minutes in 300 ul of Background Punisher (IP974G20) to block non-specific protein-protein interactions. The slides were then washed in TBS and the primary antibody applied. The primary antibody was a mouse monoclonal antibody against CD205, supplied by Leica Biosystems (Cat #NCL-L-CD205) used at a dilution of 1:80 (0.5 ug/mL) in Da Vinci Green Diluent (PD900). 300 ul of the primary antibody in diluent was applied to the slide and incubated for 30 min at room temperature. Following the primary antibody incubation, the slides were washed in TBS and 300 ul of secondary detection antibody polymer MACH 2 Mouse HRP (MHRP520) applied and incubated for 30 min at room temperature. The slides were washed in TBS and developed in 300 ul of intelliPATH FLX DAB chromogen for 5 minutes. After the chromogen is developed, the slides were washed in deionized or distilled water and lightly counterstained with Hematoxylin for 20 seconds and again rinsed in deionized water. The stained slides were then dehydrated through 3-5 minutes exchanges in graded histological grade ethanols from 70%, 90%, 95%, 100% three times, and three exchanges in xylene before mounting in Permount.

Staining was scored on a scale of 0 (negative) to 3+ (high positive), 1+ is low positive and 2+ is moderate positive. A percentage of tumor cells showing membranous staining at each intensity level was assessed by the scoring pathologist and reported (example: 0=5%, 1+=50%, 2+=35%, 3+=10%)

Patients showing greater than 50% CD205 tumor expression at at least 2+ were selected as suitable for treatment with the CD205-DM4 ADC. For the avoidance of doubt, the antibody portion of the CD205-DM4-ADC comprises antibody CD205_A1.

### Example 6: Effect of Anti-CD205 DM4 ADC on T-Cell Populations in Gastric Cancer Patient's Blood

A patient suffering from metastatic gastric cancer was administered the CD205-DM4 ADC at a dosage of 2.5 mg/kg (day 0). Blood was taken from the gastric cancer patient on days 1, 8, 15 and 21 after treatment.

### Method

All steps were performed at room temperature. 100 μl of patient blood was aliquoted into each microcentrifuge tube and antibodies added at the appropriate concentration (see table). The blood sample was stained at RT for 20 minutes and 1 ml of 1×RBC lysis buffer added. The cells were incubated for a further 15 minutes and centrifuged at 300 g for 5 minutes. The buffer was removed, and the pellet washed with 1 ml of FACS staining buffer (2% FCS+PBS+0.05% Sodium Azide).

The pellet was resuspended in 500-700 μl of FACS buffer and the sample analysed by FACS analysis.

TABLE 4

| | | Volume | |
|---|---|---|---|
| Antibody | Vendor | used | Catalog # |
| CD3-PerCp-Cy5.5 | BD Pharmingen | 5 μl | 560835 |
| CD8-FITC | BD Pharmingen | 10 μl | 557085 |
| CD4-PECY7 | BD Pharmingen | 5 μl | 560644 |
| CD205-Alexa Fluor 647 | BD Pharmingen | 5 μl | 558156 |
| PD1-BV421 | Biolegend | 5 μl | 329920 |
| CD45-PE | Thermofisher | 5 μl | 12-0459-12 |
| | Scientific | | |

### FACS Gating Strategy

Lymphocytes were initially isolated from the blood using CD45-PE antibody. The T-cells were then separated using CD3-PerCp-Cy5.5 antibodies. The separate populations of CD4+ and CD8+ cells were separated using CD4-PECY7 and CD8-FITC respectively. Subsequently the CD4+ and CD8+ cells were screened for CD205 expression and PD1 expression using CD205-Alexa Fluor 647 and PD1-BV421.

### Results

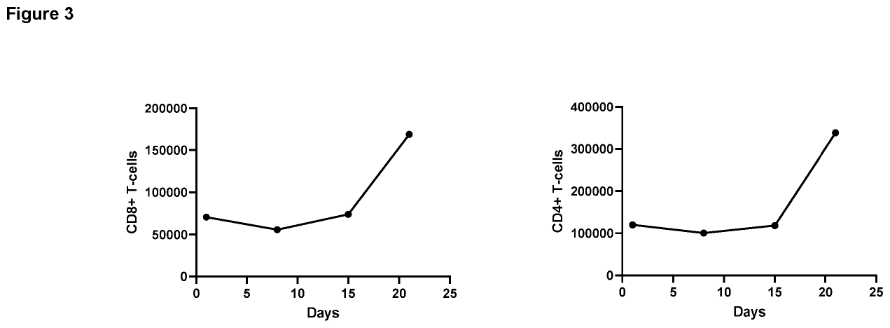

In FIG. 3 the left hand panel shows the three-fold increase in number of CD8+ T-cells present in the patient's blood between day 8 and day 21 of the 21 day time course after administration of the CD205-DM4 ADC drug. The right hand panel shows the 3.4 fold increase in the number of CD4+ T-cells present in the patient's blood between day 8 and day 21 of the 21 day time course after administration of the CD205-DM4 ADC drug. As can be seen the numbers of CD8+ and CD4+ T-cells remains relatively constant until Day 15. After this, the levels of T-cells show a rapid ˜3-fold increase between days 15 and 21.

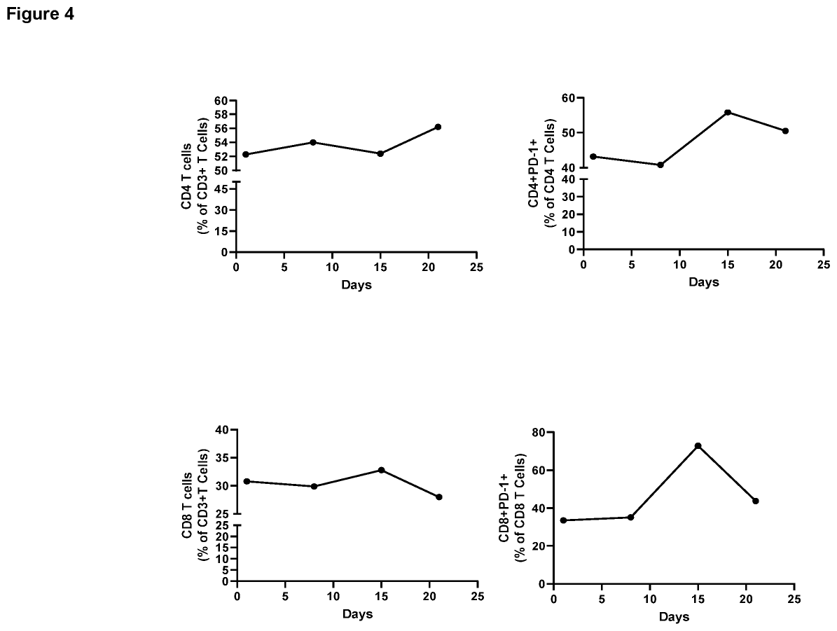

FIG. 4 shows in the left hand panels that the proportion of CD4+ and CD8+ T-cells as a percentage of the total T-cell population remains relatively constant over the time course.

The right-hand panels show the percentage of CD4+ and CD8+ T-cells that are also PD1+. As can be seen for both CD4+ and CD8+ the percentage of PD1 positive T-cells rose rapidly from day 8 and peaked at day 15.

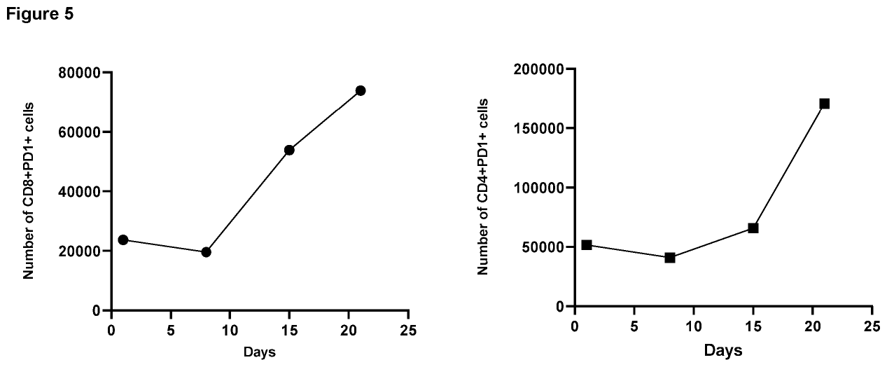

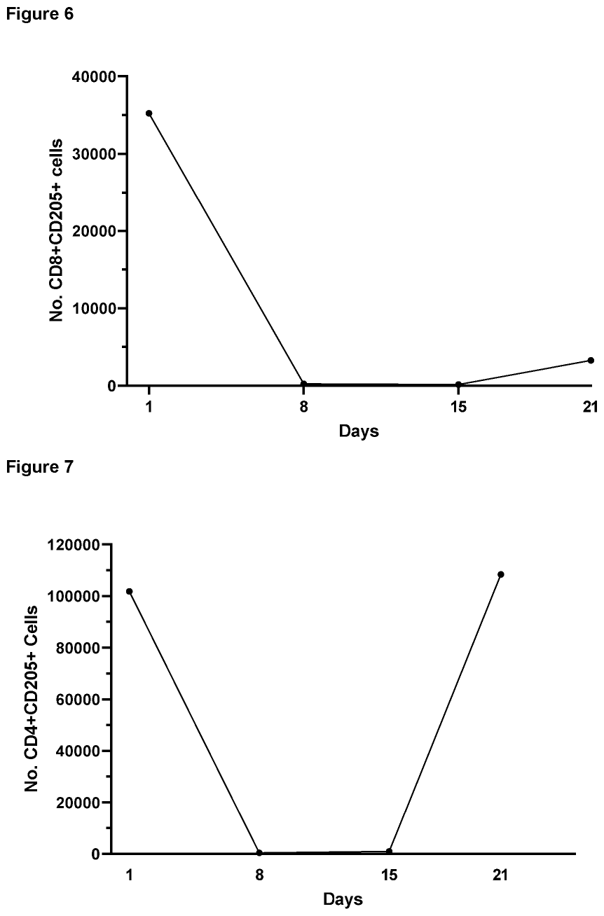

FIG. 5 shows in the left hand panel the change in number of CD8+ T-cells present in the patient's blood that are also PD1+ over the time course. The right hand panel shows the change in the number of CD4+ T-cells present in the patient's blood that are also PD1+ over the time course. As can be seen the numbers of CD8+ PD1+ T cells initially falls slightly but then rises ˜ 4-fold from day 8 to day 21. A similar pattern is seen for CD4+PD1+ T-cells. In contrast, FIGS. 6 and 7 show that the population of CD8+CD205+ and CD4+205+ immune cells fell dramatically to a very low level by day 8 and had not recovered even at day 21.

It has previously been reported that the CD8+CD205+ immune cells can induce Foxp3+ regulatory T cells which are known to mediate immunological self-tolerance and suppress immune responses (Yamazaki, S; et al, J. Immunol., 181(10), 6923, [2008]).

### Conclusions

The increase in the numbers of T-cells one week after the CD205-DM4 ADC induced drop in CD4+ CD205+ and CD8+ CD205+ immune modulatory cells supports the use of the CD205-DM4 ADC as a treatment modality to re-activate a patient's suppressed immune system in order to induce an immune response against the tumour. Furthermore, the increase in the numbers of PD1+ T-cells after treatment with the CD205-DM4 ADC supports the use of an immune checkpoint inhibitor PD1/PD-L1 to prevent a subsequent block of the CD205-DM4 ADC induced immune response by the tumour.

### Example 7: Effect of Anti CD205 DM4 ADC on Dendritic Cell Populations in Gastric Cancer Patient Blood

### Method

All steps were performed at room temperature. 100 μl of patient blood was aliquoted into each microcentrifuge tube and appropriate antibodies added (see table). The blood sample was stained at RT for 20 minutes and 1 ml of 1×RBC lysis buffer added. The cells were incubated for a further 15 minutes and centrifuged at 300 g for 5 minutes. The buffer was removed and the pellet washed with 1 ml of FACS staining buffer (2% FCS+PBS+0.05% Sodium Azide).

The pellet was resuspended in 500-700 μl of FACS buffer and the sample analysed by FACS analysis.

| | | Volume | |
|---|---|---|---|
| Antibody | Vendor | used | Catalog # |
| HLA-DR FITC | Biolegend | 5 μl | 327006 |
| CD205-Alexa Fluor 647 | BD Pharmingen | 5 μl | 558156 |
| CD123-PerCpCy5.5 | Biolegend | 5 μl | 306016 |
| CD11c-APC-Cy7 | Biolegend | 5 μl | 337218 |
| Lineage-BV510 | Biolegend | 10 μl | 348807 |
| PD-L1-PE | Biolegend | 5 μl | 329706 |

### FACS Gating Strategy

Dendritic Cells were initially isolated from the blood using the HLA-DR FITC and Lineage BV510 antibodies. The dendritic cells were then separated into pDCs and mDCs using CD11c (mDC) and CD123 (pDC) antibodies. The separate populations of mDCs and pDCs were subsequently screened for CD205 expression and PD-L1 expression using CD205-Alexa Fluor 647 and PD-L1-PE.

### Results

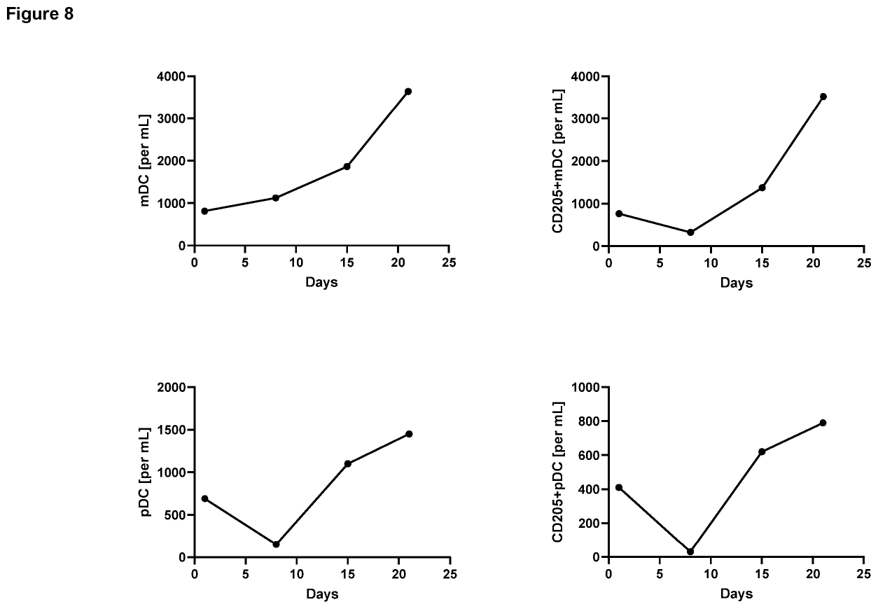

In FIG. 8 the upper left-hand panel shows that the total number of mDCs in the peripheral blood rose 4.5-fold over the 21 day time course after administration of the drug. The lower left-hand panel shows that after an initial drop the total number of peripheral pDCs doubled over the 21 day time course after administration of the drug. The right hand panels show similar patterns for CD205+ mDCs and pDCs with sharp rises seen between days 8 and 21 after an initial fallCD205.

### Example 8: Clinical Response of Gastric Cancer Patient to Treatment with 2.0-2.5 mg/kg CD205-DM4 ADC

A chemo-refractory advanced gastric patient whose tumor was MSI stable, PD-L1 negative and who had previously undergone and progressed on two lines of chemotherapy treatment (1^{st} line Docetaxel/cisplatin/5FU; 2^{nd} line Ramucirumab/Paclitaxel) and who had lymph node metastases and malignant ascites was screened by IHC for CD205 tumor expression. IHC showed that the primary tumor showed 60% 2+ CD205 expression meeting the criteria for treatment (data not shown). The patient was treated with the CD205-DM4 ADC administered at 2.5 mg/kg on a 21-day cycle. After the first cycle the dose was reduced to 2.0 mg/kg. After 3 cycles of treatment the patient was assessed. The primary gastric tumor was shown to have shrunk by ˜40% the lymph node metastases had gone as had the ascites (see Table 5). The patient was administered two further cycles of CD205-DM4 ADC followed by 1 cycle of Pembrolizumab (200 mg) (˜4 weeks after final cycle of CD205-DM4 ADC). Subsequent to treatment with Pembrolizumab, the patient was examined and found to have a complete response for the primary gastric tumour.

TABLE 5

| | | | Post Cycle 5 |
|---|---|---|---|
| | | | anti-CD205 |
| | | | Therapy and |
| | Pre-Cycle 1 | Post Cycle 3 | Post Cycle 1 |
| | anti-CD205 | anti-CD205 | Pembrolizumab |
| | Therapy | Therapy | Therapy |
| Primary Gastric | 100% | ~40% | 0% |
| Tumor | | | |
| Lymph Node | 2 | 0 | 0 |
| Mets | | | |
| Ascites | 100% | 0% | 0% |

### Example 9: Patient Blood Sample Analysis

A blood sample taken from the gastric cancer patient (Patient 1) on day 1 of cycle 1 was analysed for CD205+ expression. The patient was found to show high levels of both CD4+ and CD8+ T-cells expressing CD205 (see Table 6).

Additionally, an esophageal cancer patient (Patient 2) administered the CD205 DM4 ADC and who showed stable disease (Data not shown) was also shown to have high levels of CD205 expression on both CD4+ and CD8+ T-cells isolated from a blood sample taken on day 1 of cycle 1 of treatment.

Patients 3-5 showed low level expression of CD205 on CD4+ and CD8+ T-cells. The patients did not show the same response as Patients 1 and 2.

A further endometrial cancer patient (Patient 6) who showed complete response following two cycles of treatment with CD205 DM4 ADC and 1 cycle of treatment with Pembrolizumab was shown to have high levels of CD205 expression on both CD4+ and CD8+ T-cells.

TABLE 6

| Patient | 1 | 2 | 3 | 4 | 5 | 6 |
|---|---|---|---|---|---|---|
| CD8+CD205+(% of CD8+ cells) | 49.9% | 99.1% | 0.74% | 0.25% | 1.47% | 70.4% |
| CD4+CD205+(% of CD4+ cells) | 85.0% | 99.7% | 0.84% | 0.4% | 1.67% | 87.7% |

In light of the correlation between high levels of CD205+ T-cells in the blood of cancer patients and the anti-tumor effect of treatment with the CD205-DM4 ADC, this measure can be used to select those patients suitable for treatment with the therapy.

### Example 10: Clinical Response of Endometrial Cancer Patient to Treatment with 3.0 mg/kg CD205-DM4 ADC

An advanced endometrial cancer patient (Patient 6 above) with lung and liver metastases whose tumor was MSI stable and had low PD-L1 expression (TPS 10%; not eligible for CPI treatment) and who had previously undergone and progressed on two lines of chemotherapy treatment (1^{st} line carboplatin/taxol/herceptin; 2^{nd} line letrozole/everolimus) was screened by IHC for CD205 tumor expression. IHC showed that the primary tumor showed 100% 3+ CD205 expression meeting the criteria for treatment (data not shown). The patient was treated with the CD205-DM4 ADC administered at 3 mg/kg on a 21-day cycle. After 2 cycles of treatment the patient was administered 1 cycle of Pembrolizumab (200 mg) (˜3 weeks after final cycle of CD205-DM4 ADC). Subsequent to treatment with Pembrolizumab, the patient was examined and found to have a complete response for the primary endometrial tumour and the liver and lung metastases.

1. A method for the treatment or prophylaxis of cancer comprising administering to a patient in need thereof a therapeutically effective amount of an antibody or an antigen binding fragment thereof that modulates the population of CD205+ immune modulatory cells and a therapeutically effective amount of a composition comprising a checkpoint modulator.

2. The method according to claim 1, wherein the checkpoint modulator is directed towards a checkpoint protein selected from the group comprising PD1, PD-L1, PD-L2, CTLA-4, ICOS, TIGIT, CD28, TMIGD2, CD137, CD137L, CD27, OX40, OX40L, LAG3, VISTA, GITR, DNAM-1, CD96, 2B4, TIM-3, CEACAM, CRTAM, SLAMF6, Galectin-9, CD48, CD155, GITRL, CD40, CD40L, CD70, HVEM, B7-H7, B7-H3, B7-H4, ICOSL, CD80, CD86, BTLA, CD160, LIGHT, Adenosine A2a receptor, SIRP alpha, DC-SIGN, CD200R, DR3, TL1A, CD200, BTN2A1, CD47, IDO, TDO.

3. The method according to claim 1 or claim 2, wherein the checkpoint modulator is a PD1 or PD-L1 inhibitor, preferably PD1.

4. A method for enhancing the effectiveness of an inhibitor of PD-1/PD-L1 in a patient identified as being in need thereof, said method comprising administering to said patient (a) a therapeutically effective amount of an antibody or an antigen binding fragment thereof that modulates the population of CD205+ immune modulatory cells and (b) a composition comprising an inhibitor of PD-1/PD-L1 interactions.

5. The method according to claim 3 or claim 4, wherein the antibody or an antigen binding fragment thereof that modulates the population of CD205+ immune modulatory cells and the composition comprising the inhibitor of PD-1/PD-L1 are administered simultaneously, separately or sequentially, preferably sequentially.

6. The method according to any one of claims 1 to 5, wherein the checkpoint modulator is an antibody.

7. The method according to claim 6, wherein said antibody is an anti PD1 or PD-L1 antibody

8. The method according to claim 7, wherein said anti-PD-1 antibody is Nivolumab (MDX-1 106, Opdivo; Bristol-Myers Squibb), Pembrolizumab (MK-3475, Keytruda, lambrolizumab, BMS-936558; Merck), Cemiplimab (REGN-2810, Libtayo; Regeneron), Dostarlimab (TSR-042, Tesaro, Inc.), EH12.2H7 (ENUM-388D4, BioLegend, catalog no. 329902), Balstilimab (Agenus Inc).

9. The method according to claim 7, wherein said anti-PD-L1 antibody is Avelumab (Bavencio; EMD Serono, Pfizer), Durvalumab (Imfinzi, AstraZeneca), BMS-936559, Atezolizumab (Tecentriq, Genentech).

10. The method according to any one of claims 1 to 9, wherein the patient is administered at least 1 cycle, at least 2 cycles, at least 3 cycles, at least 4 cycles or at least 5 cycles of the antibody or an antigen binding fragment thereof that modulates the population of CD205+ immune modulatory cells prior to administration of the checkpoint modulator.

11. The method according to claim 10 wherein the patient is administered 1 to 5 cycles, 2 to 4 cycles or 2 to 3 cycles of the antibody or an antigen binding fragment thereof that modulates the population of CD205+ immune modulatory cells prior to administration of the checkpoint modulator.

12. The method of claim 10 or 11 wherein the patient is subsequently administered at least 1, at least 2, at least 3, at least 4 or at least 5 or more cycles of the checkpoint modulator.

13. The method according to any one of claims 1 to 12, wherein the checkpoint modulator is administered between 7 days and 12 weeks after administration of the antibody or antigen binding fragment thereof which modulates the population of CD205+ immune modulatory cells, preferably between 7 days and 10 weeks, or 7 days and 8 weeks, or 7 days and 6 weeks, or 7 days and 4 weeks, or 7 and 21 days or 10 and 19 days, or 12 and 16 days, or 14 and 16 days, or 19 and 28 days, more preferably 20 and 25 days, most preferably 21 and 24 days.

14. A method for increasing the anti-tumor immune response in a patient suffering from cancer comprising administering to said patient a therapeutically effective amount of an antibody or an antigen binding fragment thereof that modulates the population of CD205+ immune modulatory cells.

15. The method according to claim 14 wherein the anti-tumor immune response is an immune cell mediated tumour specific response.

16. The method according to claim 14 or claim 15, wherein the anti-tumor immune response is a NK cell mediated tumour specific response.

17. The method according to claim 14 or 15, wherein the anti-tumor immune response is a T-cell mediated tumour specific response.

18. A method for increasing the number of T-cells in a patient suffering from cancer comprising administering to said patient a therapeutically effective amount of an antibody or an antigen binding fragment thereof which modulates the population of CD205+ immune modulatory cells.

19. A method for reducing size of a tumor in a patient suffering from cancer comprising administering to said patient a therapeutically effective amount of an antibody or an antigen binding fragment thereof which modulates the population of CD205+ immune modulatory cells.

20. The method according to claim 19, wherein the tumor is a metastatic tumour.

21. The method of claim 20, wherein the metastatic tumor is in the lung or the liver.

22. The method according to any one of claims 1 to 21 wherein the population of CD205+ immune modulatory cells are CD8+.

23. The method according to claim 22 wherein the population of CD205+ CD8+ immune modulatory cells are depleted.

24. The method according to any one of claims 1 to 21 wherein the immune modulatory cells are pDCs and/or mDCs

25. The method according to claim 24 wherein the populations of pDCs and/or mDCs are increased.

26. The method according to claim any one of claims 1 to 21 wherein the population of CD205+ immune modulatory cells are CD4+.

27. The method according to claim 26 wherein the population of CD205+ CD4+ immune modulatory cells are depleted.

28. The method according to any one of claims 22 to 23 or 26 to 27 wherein the immune modulatory cells are T-Reg cells.

29. The method according to any one of claims 1 to 28, wherein the immune modulatory cells are immune inhibitory cells.

30. The method according to any one of claims 17 to 29, wherein the T-cells are CD8+ T-cells.

31. The method according to any one of claims 17 to 29, wherein the T-cells are CD4+ T-cells.

32. The method according to any one of claims 18 to 31, wherein said patient is simultaneously, separately, sequentially or subsequently administered a cancer vaccine.

33. The method according to any one of claims 18 to 31, wherein said patient is simultaneously, separately, sequentially or subsequently administered a bispecific antibody.

34. The method according to claim 33 wherein said bispecific antibody is a T-cell engager (BiTE).

35. The method according to claim 33 or claim 34, wherein said bispecific antibody comprises a first binding domain which binds to CD3.

36. The method according to any one of claims 33 to 35 wherein said bispecific antibody comprises a second binding domain which binds to tumor specific antigen.

37. The method according to any one of claims 1 to 36, wherein said patient is refractory to, or whose cancer has progressed on, at least one chemotherapy.

38. The method according to any one of claims 1 to 37, wherein said patient is refractory to checkpoint modulator therapy.

39. The method according to any one of claims 1 to 38, wherein said patient is ineligible for checkpoint modulator therapy.

40. The method according to claim 39, wherein the checkpoint modulator therapy is PD1 therapy.

41. The method according to any one of claims 1 to 40, wherein said cancer is PDL1 negative or low.

42. The method according to claim 41, wherein said patient has a cancer having less than 50%, less than 40%, less than 30%, less than 20%, less than 10%, less than 5%, less than 4%, less than 3%, less than 2% or less than 1% PD-L1 expression.

43. The method according to any one of claims 1 to 42 wherein said cancer is MSI stable.

44. The method according to any preceding claim wherein, at least 20%, least 30%, least 40%, least 50%, least 60%, least 70%, least 80%, or more, of the CD8+ cells in a blood sample previously isolated from said patient are CD205+.

45. The method according to any preceding claim wherein, at least 20%, least 30%, least 40%, least 50%, least 60%, least 70%, least 80%, or more, of the CD4+ cells in a blood sample previously isolated from said patient are CD205+.

46. The method according to any preceding claim wherein, at least 20%, least 30%, least 40%, least 50%, least 60%, least 70%, least 80%, or more, of the pDCs and/or mDCs in a blood sample previously isolated from said patient are CD205+.

47. The method according to any preceding claim, wherein the antibody or antigen binding portion thereof binds to CD205.

48. The method according to any preceding claim, wherein the antibody or antigen binding portion thereof binds to CD205 and comprises:
a heavy chain variable region comprising:
iii) a first vhCDR comprising SEQ ID NO: 5;
ii) a second vhCDR comprising SEQ ID NO: 6;
iii) a third vhCDR comprising SEQ ID NO: 7; and
a light chain variable region comprising:
iii) a first vlCDR comprising SEQ ID NO: 8;
ii) a second vlCDR comprising SEQ ID NO: 9;
iii) a third vlCDR comprising SEQ ID NO: 10;
wherein optionally any one or more of the above SEQ ID Nos independently comprise one or two amino acid substitutions, preferably conservative substitutions.

49. The method according to any preceding claim, wherein the antibody or an antigen-binding portion thereof comprises a heavy chain variable region having at least 80%, 85%, 90%, 95% or 99% amino acid sequence identity to SEQ ID NO: 1 and a light chain variable region having at least 80%, 85%, 90%, 95% or 99% amino acid sequence identity to SEQ ID NO: 2.

50. The method according to any preceding claim, wherein the antibody which binds to CD205 comprises
(iii) a heavy chain having at least 80%, 85%, 90%, 95%, 99% or 100% amino acid sequence identity to SEQ ID NO: 100; and
(ii) a light chain having at least 80%, 85%, 90%, 95%, 99% or 100% amino acid sequence identity to SEQ ID NO: 101.

51. The method according to any preceding claim, wherein the antibody or an antigen-binding portion thereof further comprises a covalently-attached moiety.

52. The method according to claim 51, wherein said moiety is a drug.

53. The method according to claim 52, wherein said drug is selected from the group consisting of a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an amatoxin, CC1065 or taxol and derivatives thereof.

54. The method according to claim 53, wherein said drug is a maytansinoid selected from the group consisting of DM4 and DM1, preferably DM4.

55. The method according to any preceding claim, wherein said cancer is a CD205 positive cancer.

56. The method according to any preceding claim, wherein said cancer is selected from the group consisting of gastric cancer, endometrial cancer, esophageal cancer, lung cancer, ovarian cancer, gastroesophageal junction cancer, pancreatic cancer, breast cancer, colorectal cancer, skin cancer, thyroid cancer, kidney cancer, liver cancer, head and neck cancer, bladder cancer, leukaemia, preferably acute myeloid leukaemia or chronic lymphocytic leukaemia, myeloma, preferably multiple myeloma and lymphoma, preferably diffuse large B-cell lymphoma (DLBCL), B-Cell Lymphoma, Follicular Lymphoma, Mantle Cell Lymphoma, Lymphoma of Mucosa-Associated Lymphoid Tissue (MALT), T-Cell/Histiocyte-Rich B-Cell Lymphoma, Burkitt's Lymphoma, Lymphoplasmacytic Lymphoma, Small Lymphocytic Lymphoma, Marginal Zone Lymphoma, T Cell Lymphoma, Peripheral T-Cell Lymphoma, Anaplastic Large Cell Lymphoma and AngioImmunoblastic T-Cell Lymphoma.

57. The method according to claim 56, wherein the cancer is selected from the group comprising: gastric cancer, endometrial cancer, esophageal cancer, lung cancer, ovarian cancer, gastroesophageal junction cancer, cancer breast cancer, bladder cancer, and renal cancer.

58. The method according to any preceding claim, wherein the patient is a human.

59. A pharmaceutical combination comprising:
iii) an anti CD205 antibody or antigen binding portion thereof, said antibody comprising:
a heavy chain variable region comprising:
iii) a first vhCDR comprising SEQ ID NO: 5;
ii) a second vhCDR comprising SEQ ID NO: 6;
iii) a third vhCDR comprising SEQ ID NO: 7; and
a light chain variable region comprising:
iii) a first vlCDR comprising SEQ ID NO: 8;
ii) a second vlCDR comprising SEQ ID NO: 9;
iii) a third vlCDR comprising SEQ ID NO: 10; and
b) a checkpoint modulator.

60. The pharmaceutical combination according to claim 59, wherein the pharmaceutical combination is in the form of a combined preparation for simultaneous, separate or sequential use, preferably sequential.

61. The pharmaceutical combination according to claim 59 or claim 60, wherein the checkpoint modulator is a PD1/PD-L1 inhibitor, preferably the PD1/PD-L1 inhibitor is an antibody.

62. The pharmaceutical combination according to claim 61, wherein the PD1/PD-L1 inhibitor is selected from the list comprising Nivolumab (MDX-1 106, Opdivo; Bristol-Myers Squibb), Pembrolizumab (MK-3475, Keytruda, lambrolizumab, BMS-936558; Merck), Dostarlimab (TSR-042 Tesaro, Inc.), Cemiplimab (REGN-2810, Libtayo; Regeneron), EH12.2H7 (BioLegend, catalog no. 329902), Balstilimab (Agenus Inc), Avelumab (Bavencio; EMD Serono, Pfizer), Durvalumab (Imfinzi, AstraZeneca), BMS-936559, Atezolizumab (Tecentriq, Genentech), or an equivalent thereto.

63. The pharmaceutical combination according to any one of claims 59 to 62, wherein the anti CD205 antibody or an antigen-binding portion thereof comprises a heavy chain variable region having at least 80%, 85%, 90%, 95% or 99% amino acid sequence identity to SEQ ID NO: 1 and a light chain variable region having at least 80%, 85%, 90%, 95% or 99% amino acid sequence identity to SEQ ID NO: 2.

64. The pharmaceutical combination according to any one of claims 59 to 63, wherein antibody which binds to CD205 comprises;
(i) a heavy chain having at least 80%, 85%, 90%, 95%, 99% or 100% amino acid sequence identity to SEQ ID NO: 100; and
(ii) a light chain having at least 80%, 85%, 90%, 95%, 99% or 100% amino acid sequence identity to SEQ ID NO: 101.

65. The pharmaceutical combination according to any one of claims 59 to 64, wherein the antibody or an antigen-binding portion thereof further comprises a covalently-attached moiety.

66. The pharmaceutical combination according to claim 65, wherein said moiety is a drug.

67. The pharmaceutical combination according to claim 66, wherein said drug is selected from the group consisting of a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an amatoxin, CC1065 or taxol and derivatives thereof.

68. The pharmaceutical combination according to claim 67, wherein said drug is a maytansinoid selected from the group consisting of DM4 and DM1, preferably DM4.

69. The pharmaceutical combination according to any one of claims 59 to 68, comprising at least one pharmaceutically acceptable diluent, excipient or carrier.

70. An antibody or antigen binding portion thereof that modulates the population of CD205+ immune modulatory cells for use in increasing the anti-tumor immune response in a patient suffering from cancer.

71. The antibody or antigen binding portion thereof for use according to claim 70 wherein the anti-tumor immune response is an immune cell mediated tumour specific response.

72. The antibody or antigen binding portion thereof for use according to claim 70 or 71, wherein the anti-tumor immune response is a NK cell mediated tumour specific response.

73. The antibody or antigen binding portion thereof for use according to any one of claims 70 to 72, wherein the anti-tumor immune response is a T-cell mediated tumour specific response.

74. An antibody or antigen binding portion thereof that modulates the population of CD205+ immune modulatory cells for use in increasing the number of T-cells in a patient suffering from cancer.

75. The antibody or antigen binding portion thereof for use according to any one of claims 70 to 74 wherein the population of CD205+ immune modulatory cells are CD8+.

76. The antibody or antigen binding portion thereof for use according to claim 75 wherein the population of CD205+ CD8+ immune modulatory cells are depleted.

77. The antibody or antigen binding portion thereof for use according to any one of claims 70 to 74 wherein the immune modulatory cells are pDCs and/or mDCs

78. The antibody or antigen binding portion thereof for use according to claim 77 wherein the populations of pDCs and/or mDCs are increased.

79. The antibody or antigen binding portion thereof for use according to any one of claims 70 to 74 wherein the population of CD205+ immune modulatory cells are CD4+.

80. The antibody or antigen binding portion thereof for use according to claim 79 wherein the population of CD205+ CD4+ immune modulatory cells are depleted.

81. The antibody or antigen binding portion thereof for use according to any one of claims 75 to 76 or 79 to 80 wherein the immune modulatory cells are T-Reg cells.

82. The antibody or antigen binding portion thereof for use according to any one of claims 70 to 81, wherein the immune modulatory cells are immune inhibitory cells.

83. The antibody or antigen binding portion thereof for use according to any one of claim 73 to claim 82 wherein the T-cells are CD8+ T-cells.

84. The antibody or antigen binding portion thereof for use according to any one of claim 73 to claim 82, wherein the T-cells are CD4+ T-cells.

85. The antibody or antigen binding portion thereof for use according to any one of claims 70 to 84, wherein said patient is simultaneously, separately, sequentially or subsequently administered a cancer vaccine.

86. The antibody or antigen binding portion thereof for use according to any one of claims 70 to 84, wherein said patient is simultaneously, separately, sequentially or subsequently administered a bispecific antibody.

87. The antibody or antigen binding portion thereof for use according to claim 86 wherein said bispecific antibody is a T-cell engager (BITE).

88. The antibody or antigen binding portion thereof for use according to claim 86 or claim 82, wherein said bispecific antibody comprises a first binding domain which binds to CD3.

89. The antibody or antigen binding portion thereof for use according to any one of claims 86 to 88 wherein said bispecific antibody comprises a second binding domain which binds to tumor specific antigen.

90. The antibody or antigen binding portion thereof for use according to any one of claims 70 to 89, wherein said patient is refractory to, or whose cancer has progressed on at least one chemotherapy.

91. The antibody or antigen binding portion thereof for use according to any one of claims 70 to 90, wherein said patient is refractory to checkpoint modulator therapy.

92. The antibody or antigen binding portion thereof for use according to claim 91, wherein the checkpoint modulator therapy is PD1 inhibitor therapy.

93. The antibody or antigen binding portion thereof for use according to any one of claims 70 to 92, wherein said cancer is PDL1 negative or low.

94. The antibody or antigen binding portion thereof for use according to claim 93, wherein said cancer has less than 50%, less than 40%, less than 30%, less than 20%, less than 10%, less than 5%, less than 4%, less than 3%, less than 2% or less than 1% PD-L1 expression.

95. The antibody or antigen binding portion thereof for use according to any one of claims 70 to 94, wherein said cancer is MSI stable.

96. The antibody or antigen binding portion thereof for use according to any one of claims 70 to 95, wherein at least 20%, least 30%, least 40%, least 50%, least 60%, least 70%, least 80%, or more, of the CD8+ cells in a blood sample previously isolated from said patient are CD205+.

97. The antibody or antigen binding portion thereof for use according to any one of claims 70 to 95, wherein at least 20%, least 30%, least 40%, least 50%, least 60%, least 70%, least 80%, or more, of the CD4+ cells in a blood sample previously isolated from said patient are CD205+.

98. The antibody or antigen binding portion thereof for use according to any one of claims 70 to 95, wherein at least 20%, least 30%, least 40%, least 50%, least 60%, least 70%, least 80%, or more, of the pDCs and/or mDCs in a blood sample previously isolated from said patient are CD205+.

99. A pharmaceutical combination for use in the treatment or prophylaxis of cancer, said combination comprising; an antibody or antigen binding portion thereof that modulates the population of CD205+ immune modulatory cells; and a composition comprising a checkpoint modulator.

100. The pharmaceutical combination for use according to claim 99, wherein the checkpoint modulator is directed towards a checkpoint protein selected from the group comprising PD1, PD-L1, PD-L2, CTLA-4, ICOS, TIGIT, CD28, TMIGD2, CD137, CD137L, CD27, OX40, OX40L, LAG3, VISTA, GITR, DNAM-1, CD96, 2B4, TIM-3, CEACAM, CRTAM, SLAMF6, Galectin-9, CD48, CD155, GITRL, CD40, CD40L, CD70, HVEM, B7-H7, B7-H3, B7-H4, ICOSL, CD80, CD86, BTLA, CD160, LIGHT, Adenosine A2a receptor, SIRP alpha, DC-SIGN, CD200R, DR3, TL1A, CD200, BTN2A1, CD47, IDO, TDO.

101. The pharmaceutical combination for use according to claim 99 or 100, wherein the checkpoint modulator is a PD1 or PD-L1 inhibitor, preferably PD1.

102. A pharmaceutical combination for use in enhancing the effectiveness of an inhibitor of PD-1/PD-L1 interactions in a patient, said combination comprising; an antibody or antigen binding portion thereof that modulates the population of CD205+ immune modulatory cells; and a composition comprising an inhibitor of PD1/PD-L1 interactions.

103. The pharmaceutical combination for use according to any one of claims 99 to 102 wherein the pharmaceutical combination is in the form of a combined preparation for simultaneous, separate or sequential use, preferably sequential.

104. The pharmaceutical combination for use according to any one of claims 99 to 103, wherein the population of CD205+ immune modulatory cells are CD8+.

105. The pharmaceutical combination for use according to claim 104, wherein the population of CD205+ CD8+ immune modulatory cells are depleted.

106. The pharmaceutical combination for use according to any one of claims 99 to 103, wherein the immune modulatory cells are pDCs and/or mDCs.

107. The pharmaceutical combination for use according to claim 106 wherein the populations of pDCs and/or mDCs are increased.

108. The pharmaceutical combination for use according to any one of claims 99 to 103, wherein the population of CD205+ immune modulatory cells are CD4+.

109. The pharmaceutical combination for use according to claim 108 wherein the population of CD205+ CD4+ immune modulatory cells are depleted.

110. The pharmaceutical combination for use according to any one of claims 104 to 105 or 108 to 109 wherein the immune modulatory cells are T-Reg cells.

111. The pharmaceutical combination for use according to any one of claims 99 to 110, wherein the immune modulatory cells are immune inhibitory cells.

112. The pharmaceutical combination for use according to any one of claims 99 to 111, wherein said patient is simultaneously, separately, sequentially or subsequently administered a cancer vaccine.

113. The pharmaceutical combination for use to any one of claims 99 to 111, wherein said patient is simultaneously, separately, sequentially or subsequently administered a bispecific antibody.

114. The pharmaceutical combination for use according to claim 113 wherein said bispecific antibody is a T-cell engager (BiTE).

115. The pharmaceutical combination for use according to claim 113 or claim 114, wherein said bispecific antibody comprises a first binding domain which binds to CD3.

116. The pharmaceutical combination for use according to any one of claims 113 to 114 wherein said bispecific antibody comprises a second binding domain which binds to tumor specific antigen.

117. The pharmaceutical combination for use according to any one of claims 99 to 116, wherein the patient is administered at least 1 cycle, at least 2 cycles, at least 3 cycles, at least 4 cycles or at least 5 cycles of the antibody or an antigen binding fragment thereof that modulates the population of CD205+ immune modulatory cells prior to administration of the checkpoint modulator.

118. The pharmaceutical combination for use according to claim 117 wherein the patient is administered 1 to 5 cycles, 2 to 4 cycles or 2 to 3 cycles of the antibody or an antigen binding fragment thereof that modulates the population of CD205+ immune modulatory cells prior to administration of the checkpoint modulator.

119. The pharmaceutical combination for use of claim 117 or 118 wherein the patient is subsequently administered at least 1, at least 2, at least 3, at least 4 or at least 5 or more cycles of the checkpoint modulator.

120. The pharmaceutical combination for use according to any one of claims 99 to 119, wherein the checkpoint modulator is administered between 7 days and 12 weeks after administration of the antibody or antigen binding portion thereof which binds to CD205, preferably between 7 days and 10 weeks, or 7 days and 8 weeks, or 7 days and 6 weeks, or 7 days and 4 weeks, or 7 and 21 days or 10 and 19 days, or 12 and 16 days, or 14 and 16 days, or 19 and 28 days, more preferably 20 and 25 days, most preferably 21 and 24 days.

121. The pharmaceutical combination for use according to any one of claims 99 to 120, wherein said patient is refractory to, or whose cancer has progressed on, at least one chemotherapy.

122. The pharmaceutical combination for use according to any one of claims 99 to 121, wherein said patient is refractory to checkpoint modulator therapy.

123. The pharmaceutical combination for use according to claim 122, wherein the checkpoint modulator therapy is PD1 inhibitor therapy.

124. The pharmaceutical combination for use according to any one of claims 99 to 123, wherein said cancer is PDL1 negative or low.

125. The pharmaceutical combination for use according to any one of claims 99 to 124, wherein said cancer is MSI stable.

126. The pharmaceutical combination for use according to any one of claims 99 to 125 wherein, at least 20%, least 30%, least 40%, least 50%, least 60%, least 70%, least 80%, or more, of the CD8+ cells in a blood sample previously isolated from said patient are CD205+.

127. The pharmaceutical combination for use according to any one of claims 99 to 125 wherein, at least 20%, least 30%, least 40%, least 50%, least 60%, least 70%, least 80%, or more, of the CD4+ cells in a blood sample previously isolated from said patient are CD205+.

128. The pharmaceutical combination for use according to any one of claims 99 to 125 wherein, at least 20%, least 30%, least 40%, least 50%, least 60%, least 70%, least 80%, or more, of the pDCs and/or mDCs in a blood sample previously isolated from said patient are CD205+.

129. The antibody or antigen binding portion thereof or pharmaceutical combination for use according to any one of claims 70 to 128, wherein the antibody or antigen binding portion thereof binds to CD205.

130. The antibody or antigen binding portion thereof or pharmaceutical combination for use according to any one of claims 70 to 129, wherein the antibody or antigen binding portion thereof which binds to CD205 comprises:
a heavy chain variable region comprising:
i) a first vhCDR comprising SEQ ID NO: 5;
ii) a second vhCDR comprising SEQ ID NO: 6; and
iii) a third vhCDR comprising SEQ ID NO: 7; and
a light chain variable region comprising:
i) a first vlCDR comprising SEQ ID NO: 8;
ii) a second vlCDR comprising SEQ ID NO: 9; and
iii) a third vlCDR comprising SEQ ID NO: 10
wherein optionally any one or more of the above SEQ ID NOs independently comprise one or two amino acid substitutions, preferably conservative substitutions.

131. The antibody or antigen binding portion thereof or pharmaceutical combination for use according to any one of claim 70 to claim 130, wherein the antibody or an antigen-binding portion thereof which binds to CD205 comprises a heavy chain variable region having at least 80%, 85%, 90%, 95% or 99% amino acid sequence identity to SEQ ID NO: 1 and a light chain variable region having at least 80%, 85%, 90%, 95% or 99% amino acid sequence identity to SEQ ID NO: 2.

132. The antibody or pharmaceutical combination for use according to any one of claims 70 to 131, wherein antibody which binds to CD205 comprises;
(i) a heavy chain having at least 80%, 85%, 90%, 95%, 99% or 100% amino acid sequence identity to SEQ ID NO: 100; and
(ii) a light chain having at least 80%, 85%, 90%, 95%, 99% or 100% amino acid sequence identity to SEQ ID NO: 101.

133. The antibody or antigen binding portion thereof or pharmaceutical combination for use according to any one of claims 70 to 132, wherein the antibody or an antigen-binding portion thereof further comprises a covalently-attached moiety.

134. The antibody or antigen binding portion thereof or pharmaceutical combination for use according to claim 133, wherein said moiety is a drug.

135. The antibody or antigen binding portion thereof or pharmaceutical combination for use according to claim 134, wherein said drug is selected from the group consisting of a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an amatoxin, CC1065 or taxol and derivatives thereof.

136. The antibody or antigen binding portion thereof or pharmaceutical combination for use according to claim 135, wherein said drug is a maytansinoid selected from the group consisting of DM4 and DM1, preferably DM4.

137. The pharmaceutical combination for use according to any one of claims 99 to 136, wherein the checkpoint modulator is an antibody.

138. The pharmaceutical combination for use according to claim 137 wherein said antibody is an anti PD1 or PD-L1 antibody.

139. The pharmaceutical combination for use according to claim 138, wherein said anti-PD-1 antibody is Nivolumab (MDX-1 106, Opdivo; Bristol-Myers Squibb), Pembrolizumab (MK-3475, Keytruda, lambrolizumab, BMS-936558; Merck), Dostarlimab (TSR-042 Tesaro, Inc.), Cemiplimab (REGN2810 Regeneron Pharmaceuticals), EH12.2H7 (BioLegend, catalog no. 329902), Balstilimab (Agenus Inc).

140. The pharmaceutical combination for use according to claim 138, wherein said anti-PD-L1 antibody is Avelumab (Bavencio; EMD Serono, Pfizer), Durvalumab (Imfinzi, AstraZeneca), BMS-936559, Atezolizumab (Tecentriq, Genentech).

141. The antibody or antigen binding portion thereof or pharmaceutical combination for use according to any one of claims 70 to 140, wherein said cancer is a CD205 positive cancer.

142. The antibody or antigen binding portion thereof or pharmaceutical combination for use according to any one of claims 70 to 141, wherein said cancer is selected from the group consisting of gastric cancer, endometrial cancer, esophageal cancer, lung cancer, ovarian cancer, gastroesophageal junction cancer, pancreatic cancer, breast cancer, colorectal cancer, skin cancer, thyroid cancer, kidney cancer, liver cancer, head and neck cancer, bladder cancer, leukaemia, preferably acute myeloid leukaemia or chronic lymphocytic leukaemia, myeloma, preferably multiple myeloma and lymphoma, preferably diffuse large B-cell lymphoma (DLBCL), B-Cell Lymphoma, Follicular Lymphoma, Mantle Cell Lymphoma, Lymphoma of Mucosa-Associated Lymphoid Tissue (MALT), T-Cell/Histiocyte-Rich B-Cell Lymphoma, Burkitt's Lymphoma, Lymphoplasmacytic Lymphoma, Small Lymphocytic Lymphoma, Marginal Zone Lymphoma, T Cell Lymphoma, Peripheral T-Cell Lymphoma, Anaplastic Large Cell Lymphoma and AngioImmunoblastic T-Cell Lymphoma.

143. The antibody or antigen binding portion thereof or pharmaceutical combination for use according to claim 142, wherein the cancer is selected from the group comprising: gastric cancer, endometrial cancer, esophageal cancer, lung cancer, ovarian cancer, gastroesophageal junction cancer, breast cancer, bladder cancer, and renal cancer.

144. The antibody or antigen binding portion thereof or pharmaceutical combination for use according to any one of claims 70 to 143, wherein the patient is a human.

145. A method for selecting a patient suitable for therapy with an antibody or antigen binding fragment thereof which binds to CD205, wherein said patient is suffering from cancer, said method comprising:
identifying a patient wherein at least 20% of the CD8+ cells in a blood sample previously isolated from said patient are CD205+ and administering a therapeutically effective amount of an anti CD205 antibody or antigen binding fragment thereof to said patient.

146. An in vitro method for selecting a patient suitable for treatment with an antibody or antigen binding fragment thereof which binds to CD205 comprising:
a. determining the percentage of CD8+ cells in a blood sample previously isolated from said patient that are CD205+ cells; and
b. selecting the patient for treatment with the antibody or antigen binding fragment thereof which binds to CD205 if at least 20% of the CD8+ cells in the blood sample CD205+.

147. The in vitro method of claim 146, further comprising the step of administering to said patient a therapeutically effective amount of said antibody or antigen binding fragment thereof which binds to CD205.

148. A method for determining the efficacy of an antibody or antigen binding fragment thereof which binds to CD205 in the treatment of cancer in a patient, said method comprising
a. obtaining a blood sample from said patient,
b. identifying whether at least 20% of the CD8+ cells in the blood sample are CD205+.

149. The method according to claim 148, further comprising the step of administering to said patient a therapeutically effective amount of an antibody or antigen binding fragment thereof which binds to CD205 if at least 20% of the CD8+ cells in the blood sample are CD205+.

150. The method according to any one of claims 145 to 149 wherein at least 30%, at least 40%, at least 50%, at least 60%, at least 70%, at least 80%, at least 90%, at least 95%, at least 96%, at least 97%, at least 98%, at least 99% of said patient's CD8+ cells are CD205+.

151. A method for selecting a patient suitable for therapy with an antibody or antigen binding fragment thereof which binds to CD205, wherein said patient is suffering from cancer, said method comprising:
identifying a patient wherein at least 20% of the CD4+ cells in a blood sample previously isolated from said patient are CD205+ and administering a therapeutically effective amount of an anti CD205 antibody or antigen binding fragment thereof to said patient.

152. An in vitro method for selecting a patient suitable for treatment with an antibody or antigen binding fragment thereof which binds to CD205 comprising:
a. determining the percentage of CD4+ cells in a blood sample previously isolated from said patient that are CD205+ cells; and
b. selecting the patient for treatment with the antibody or antigen binding fragment thereof which binds to CD205 if at least 20% of the CD4+ cells in the blood sample CD205+.

153. The in vitro method of claim 152, further comprising the step of administering to said patient a therapeutically effective amount of said antibody or antigen binding fragment thereof which binds to CD205.

154. A method for determining the efficacy of an antibody or antigen binding fragment thereof which binds to CD205 in the treatment of cancer in a patient, said method comprising
a. obtaining a blood sample from said patient,
b. identifying whether at least 20% of the CD4+ cells in the blood sample are CD205+.

155. The method according to claim 154, further comprising the step of administering to said patient a therapeutically effective amount of an antibody or antigen binding fragment thereof which binds to CD205 if at least 20% of the CD4+ cells in the blood sample are CD205+.

156. The method according to any one of claims 151 to 155 wherein at least 30%, at least 40%, at least 50%, at least 60%, at least 70%, at least 80%, at least 90%, at least 95%, at least 96%, at least 97%, at least 98%, at least 99% of said patient's CD4+ cells are CD205+.

157. A method for selecting a patient suitable for therapy with an antibody or antigen binding fragment thereof which binds to CD205, wherein said patient is suffering from cancer, said method comprising:
identifying a patient wherein at least 20% of the CD8+ and CD4+ cells in a blood sample previously isolated from said patient are CD205+ and administering to said patient a therapeutically effective amount of an anti CD205 antibody or antigen binding fragment thereof to said patient.

158. An in vitro method for selecting a patient suitable for treatment with an antibody or antigen binding fragment thereof which binds to CD205 comprising:
a. determining the percentage of CD8+ and CD4+ cells in a blood sample previously isolated from said patient that are CD205+ cells; and
b. selecting the patient for treatment with the antibody or antigen binding fragment thereof which binds to CD205 if at least 20% of the CD8+ and CD4+ cells in the blood sample CD205+.

159. The in vitro method of claim 158, further comprising the step of administering to said patient a therapeutically effective amount of an antibody or antigen binding fragment thereof which binds to CD205.

160. A method for determining the efficacy of an antibody or antigen binding fragment thereof which binds to CD205 in the treatment of cancer in a patient, said method comprising
a. obtaining a blood sample from said patient,
b. identifying whether at least 20% of the CD8+ and CD4+ cells in the blood sample are CD205+.

161. The method according to claim 160, further comprising the step of administering to said patient a therapeutically effective amount of an antibody or antigen binding fragment thereof which binds to CD205 if at least 20% of the CD8+ and CD4+ cells in the blood sample are CD205+.

162. The method according to any one of claims 157 to 161 wherein at least 30%, at least 40%, at least 50%, at least 60%, at least 70%, at least 80%, at least 90%, at least 95%, at least 96%, at least 97%, at least 98%, at least 99% of said patient's CD8+ and CD4+ cells are CD205+.

163. A method for the treatment or prophylaxis of cancer comprising, identifying a patient wherein at least 20% of the CD8+ cells in a blood sample previously isolated from said patient are CD205+ and administering to said patient a therapeutically effective amount of an antibody or antigen binding fragment thereof which binds to CD205.

164. The method according to claim 163, wherein at least 30%, at least 40%, at least 50%, at least 60%, at least 70%, at least 80%, at least 90%, at least 95%, at least 96%, at least 97%, at least 98%, at least 99% of said patient's CD8+ cells are CD205+.

165. A method for the treatment or prophylaxis of cancer comprising, identifying a patient wherein at least 20% of the CD4+ cells in a blood sample previously isolated from said patient are CD205+ and administering to said patient a therapeutically effective amount of an antibody or antigen binding fragment thereof which binds to CD205.

166. The method according to claim 165, wherein at least 30%, at least 40%, at least 50%, at least 60%, at least 70%, at least 80%, at least 90%, at least 95%, at least 96%, at least 97%, at least 98%, at least 99% of said patient's CD4+ cells are CD205+.

167. A method for the treatment or prophylaxis of cancer comprising identifying a patient wherein at least 20% of the CD8+ cells and CD4+ cells in a blood sample previously isolated from said patient are CD205+ and administering to said patient a therapeutically effective amount of an antibody or antigen binding fragment thereof which binds to CD205.

168. The method according to claim 167, wherein at least 30%, at least 40%, at least 50%, at least 60%, at least 70%, at least 80%, at least 90%, at least 95%, at least 96%, at least 97%, at least 98%, at least 99% of said patient's CD8+ and CD4+ cells are CD205+.

169. A treatment method comprising:
(a) calculating the percentage of CD4+ and/or CD8+ cells that are CD205+ in a blood sample previously isolated from a patient diagnosed with cancer to identify the patient as having a responder phenotype; and
(b) administering a therapeutically effective amount of an antibody or antigen binding fragment thereof which binds to CD205 to the patient having a responder phenotype.

170. The treatment method according to claim 169, wherein at least 20%, at least 30%, at least 40%, at least 50%, at least 60%, at least 70%, at least 80%, at least 90%, at least 95%, at least 96%, at least 97%, at least 98%, at least 99% of the CD4+ and/or CD8+ cells in the blood sample previously isolated from a patient diagnosed with cancer are CD205+ positive.

171. The method according to any one of claims 145 to 170, comprising the further step of subsequently administering to said patient a checkpoint modulator.

172. The method according to claim 171, wherein the checkpoint modulator is directed towards a checkpoint protein selected from the group comprising PD1, PD-L1, PD-L2, CTLA-4, ICOS, TIGIT, CD28, TMIGD2, CD137, CD137L, CD27, OX40, OX40L, LAG3, VISTA, GITR, DNAM-1, CD96, 2B4, TIM-3, CEACAM, CRTAM, SLAMF6, Galectin-9, CD48, CD155, GITRL, CD40, CD40L, CD70, HVEM, B7-H7, B7-H3, B7-H4, ICOSL, CD80, CD86, BTLA, CD160, LIGHT, Adenosine A2a receptor, SIRP alpha, DC-SIGN, CD200R, DR3, TL1A, CD200, BTN2A1, CD47, IDO, TDO.

173. The method according to claim 172, wherein the checkpoint modulator is an antibody.

174. The method according to claim 173, wherein said antibody is a PD1 or PD-L1 inhibitor, preferably PD1.

175. The method according to claim 174, wherein said anti-PD-1 antibody is Nivolumab (MDX-1 106, Opdivo; Bristol-Myers Squibb), Pembrolizumab (MK-3475, Keytruda, lambrolizumab, BMS-936558; Merck), Cemiplimab (REGN-2810, Libtayo; Regeneron), Dostarlimab (TSR-042, Tesaro, Inc.), EH12.2H7 (ENUM-388D4, BioLegend, catalog no. 329902), Balstilimab (Agenus Inc).

176. The method according to claim 174, wherein said anti-PD-L1 antibody is Avelumab (Bavencio; EMD Serono, Pfizer), Durvalumab (Imfinzi, AstraZeneca), BMS-936559, Atezolizumab (Tecentriq, Genentech).

177. The method according to any one of claims 145 to 176, wherein said patient is simultaneously, separately, sequentially or subsequently administered a cancer vaccine.

178. The method according to any one of claims 145 to 176, wherein said patient is simultaneously, separately, sequentially or subsequently administered a bispecific antibody.

179. The method according to claim 178 wherein said bispecific antibody is a T-cell engager (BiTE).

180. The method according to claim 178 or claim 179, wherein said bispecific antibody comprises a first binding domain which binds to CD3.

181. The method according to any one of claims 178 to 180 wherein said bispecific antibody comprises a second binding domain which binds to tumor specific antigen.

182. The method according to any one of claims 145 to 181, wherein said patient is refractory to, or whose cancer has progressed on, at least one previously line of chemotherapy.

183. The method according to any one of claims 145 to 182, wherein said patient is refractory to checkpoint modulator therapy.

184. The method according to claim 183, wherein the checkpoint modulator therapy is PD1 therapy.

185. The method according to claim any one of claims 145 to 184, wherein said cancer is PDL1 negative or low.

186. The method according to any one of claims 145 to 185 wherein said cancer is MSI stable.

187. The method according to any one of claims 145 to 186, wherein the patient is administered at least 1 cycle, at least 2 cycles, at least 3 cycles, at least 4 cycles or at least 5 cycles of the antibody or an antigen binding fragment thereof that modulates the population of CD205+ immune modulatory cells prior to administration of the checkpoint inhibitor.

188. The method according to claim 187 wherein the patient is administered 1 to 5 cycles, 2 to 4 cycles or 2 to 3 cycles of the antibody or an antigen binding fragment thereof that modulates the population of CD205+ immune modulatory cells prior to administration of the checkpoint modulator.

189. The method according to any one of claims 171 to 188 wherein the patient is administered at least 1, at least 2, at least 3, at least 4 or at least 5 or more cycles of the checkpoint modulator.

190. The method according to any one of claims 171 to 189, wherein the checkpoint modulator is administered between 7 days and 12 weeks after administration of the antibody or antigen binding portion thereof which binds to CD205, preferably between 7 days and 10 weeks, or 7 days and 8 weeks, or 7 days and 6 weeks, or 7 days and 4 weeks, or 7 and 21 days or 10 and 19 days, or 12 and 16 days, or 14 and 16 days, or 19 and 28 days, more preferably 20 and 25 days, most preferably 21 and 24 days.


SEQUENCE LISTING (120 sequences)

SEQ ID NO: 1 - protein, 119 aa, Homo sapiens
EVQLVESGGGLVKPGGSLRLSCAASGFTYSNAWMSWVRQAPGKGLEWVGRIKSKTDGGTTDYAAPVQGRFTISRDDSKNTLYLQMNSLKTEDTAVYYCTIFGVVSFDYWGQGTLVTVSS

SEQ ID NO: 2 - protein, 108 aa, Homo sapiens
DVQMTQSPSSLSASVGDRVTITCRASQSISDYLSWYQQRPGKAPNLLIYAASNLKTGVPSRFSGSGSGTDFTLTISTLQPEDFATYYCQQSYRSPWTFGQGTKVEIKR

SEQ ID NO: 3 - DNA, 357 bp, Homo sapiens
gaggtgcagctggtggagtctgggggaggcttggtaaagccgggggggtcccttagactctcctgtgcagcctctggcttcacttacagtaacgcctggatgagctgggtccgccaggctccagggaaggggctggagtgggttggccgtattaaaagcaaaactgatggtgggacaacagactacgctgcacccgtgcaaggcagattcaccatctcaagagatgattcaaaaaacacgctgtatctgcaaatgaacagcctgaaaaccgaggacacagccgtgtattactgtacgatttttggagtggttagctttgactactggggccagggaaccctggtcaccgtctcctca

SEQ ID NO: 4 - DNA, 324 bp, Homo sapiens
gacgtccagatgacccagtctccatcctccctgtctgcatctgttggagacagagtcaccatcacttgccgggcaagtcagagcattagcgactatttaagttggtatcagcagagaccagggaaagcccctaacctcctgatctatgctgcatccaatttaaagactggggtcccatcaaggttcagtggcagtggatctgggacagatttcactctcaccatcagcactctgcaacctgaagattttgcaacgtactactgtcaacagagttacaggtccccgtggacgttcggccaagggaccaaggtggaaatcaaacga

SEQ ID NO: 5 - protein, 5 aa, Homo sapiens
NAWMS

SEQ ID NO: 6 - protein, 19 aa, Homo sapiens
RIKSKTDGGTTDYAAPVQG

SEQ ID NO: 7 - protein, 8 aa, Homo sapiens
FGVVSFDY

SEQ ID NO: 8 - protein, 11 aa, Homo sapiens
RASQSISDYLS

SEQ ID NO: 9 - protein, 7 aa, Homo sapiens
AASNLKT

SEQ ID NO: 10 - protein, 9 aa, Homo sapiens
QQSYRSPWT

SEQ ID NO: 11 - protein, 1722 aa, Homo sapiens
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNKSLMWFDKTPLSYTHWRAGRPTIKNEKFLAGLSTDGFWDIQTFKVIEEAVYFHQHSILACKIEMVDYKEEYNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 12 - protein, 30 aa, Homo sapiens
EVQLVESGGGLVKPGGSLRLSCAASGFTYS

SEQ ID NO: 13 - protein, 14 aa, Homo sapiens
WVRQAPGKGLEWVG

SEQ ID NO: 14 - protein, 32 aa, Homo sapiens
RFTISRDDSKNTLYLQMNSLKTEDTAVYYCTI

SEQ ID NO: 15 - protein, 11 aa, Homo sapiens
WGQGTLVTVSS

SEQ ID NO: 16 - protein, 23 aa, Homo sapiens
DVQMTQSPSSLSASVGDRVTITC

SEQ ID NO: 17 - protein, 15 aa, Homo sapiens
WYQQRPGKAPNLLIY

SEQ ID NO: 18 - protein, 32 aa, Homo sapiens
GVPSRFSGSGSGTDFTLTISTLQPEDFATYYC

SEQ ID NO: 19 - protein, 11 aa, Homo sapiens
FGQGTKVEIKR

SEQ ID NO: 20 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 5 aa, Homo sapiens
NSGMH

SEQ ID NO: 23 - protein, 17 aa, Homo sapiens
VIWYDGSKRYYADSVKG

SEQ ID NO: 24 - protein, 4 aa, Homo sapiens
NDDY

SEQ ID NO: 25 - protein, 11 aa, Homo sapiens
RASQSVSSYLA

SEQ ID NO: 26 - protein, 7 aa, Homo sapiens
DASNRAT

SEQ ID NO: 27 - protein, 9 aa, Homo sapiens
QQSSNWPRT

SEQ ID NO: 28 - protein, 447 aa, Mus musculus
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 218 aa, Mus musculus
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 5 aa, Mus musculus
NYYMY

SEQ ID NO: 31 - protein, 17 aa, Mus musculus
GINPSNGGTNFNEKFKN

SEQ ID NO: 32 - protein, 11 aa, Mus musculus
RDYRFDMGFDY

SEQ ID NO: 33 - protein, 15 aa, Mus musculus
RASKGVSTSGYSYLH

SEQ ID NO: 34 - protein, 7 aa, Mus musculus
LASYLES

SEQ ID NO: 35 - protein, 9 aa, Mus musculus
GHSRDLPLT

SEQ ID NO: 36 - protein, 444 aa, Homo sapiens
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 214 aa, Homo sapiens
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 8 aa, Homo sapiens
GFTFSNFG

SEQ ID NO: 39 - protein, 8 aa, Homo sapiens
ISGGGRDT

SEQ ID NO: 40 - protein, 10 aa, Homo sapiens
VKWGNIYFDY

SEQ ID NO: 41 - protein, 6 aa, Homo sapiens
LSINTF

SEQ ID NO: 42 - protein, 3 aa, Homo sapiens
AAS

SEQ ID NO: 43 - protein, 9 aa, Homo sapiens
QQSSNTPFT

SEQ ID NO: 44 - protein, 443 aa, Artificial Sequence
  Humanized antibody sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 214 aa, Artificial Sequence
  Humanized antibody sequence
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 5 aa, Artificial Sequence
  Humanized sequence
SYDMS

SEQ ID NO: 47 - protein, 17 aa, Artificial Sequence
  Humanized sequence
TISGGGSYTYYQDSVKG

SEQ ID NO: 48 - protein, 7 aa, Artificial Sequence
  Humanized sequence
PYYAMDY

SEQ ID NO: 49 - protein, 11 aa, Artificial Sequence
  Humanized sequence
KASQDVGTAVA

SEQ ID NO: 50 - protein, 7 aa, Artificial Sequence
  Humanized sequence
WASTLHT

SEQ ID NO: 51 - protein, 9 aa, Artificial Sequence
  Humanized sequence
QHYSSYPWT

SEQ ID NO: 52 - protein, 120 aa, Artificial Sequence
  Humanized sequence
EVQLVQSGAEVKKPGASVKVSCKASGYTFTDYEMHWVRQAPGQRLEWMGVIDPGTGGTAYNQKFQGRVTITADKSASTAYMELSSLRSEDTAVYYCTSEKFGSNYYFDYWGQGTLVTVSS

SEQ ID NO: 53 - protein, 112 aa, Artificial Sequence
  Humanized sequence
DIVMTQTPLSSPVTLGQPASISCRSSQTIVHSDGNTYLEWYQQRPGQPPRLLIYKVSNRFSGVPDRFSGSGAGTDFTLKISRVEAEDVGVYYCFQGSHVPLTFGQGTKLEIK

SEQ ID NO: 54 - protein, 8 aa, Artificial Sequence
  Humanized sequence
GYTFTDYE

SEQ ID NO: 55 - protein, 9 aa, Artificial Sequence
  Humanized sequence
IDPGTGGTA

SEQ ID NO: 56 - protein, 13 aa, Artificial Sequence
  Humanized sequence
TSEKFGSNYYFDY

SEQ ID NO: 57 - protein, 11 aa, Artificial Sequence
  Humanized sequence
QTIVHSDGNTY

SEQ ID NO: 58 - protein, 3 aa, Artificial Sequence
  Humanized sequence
KVS

SEQ ID NO: 59 - protein, 9 aa, Artificial Sequence
  Humanized sequence
FQGSHVPLT

SEQ ID NO: 60 - protein, 450 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 216 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 62 - protein, 5 aa, Homo sapiens
SYIMM

SEQ ID NO: 63 - protein, 17 aa, Homo sapiens
SIYPSGGITFYADTVKG

SEQ ID NO: 64 - protein, 11 aa, Homo sapiens
IKLGTVTTVDY

SEQ ID NO: 65 - protein, 14 aa, Homo sapiens
TGTSSDVGGYNYVS

SEQ ID NO: 66 - protein, 7 aa, Homo sapiens
DVSNRPS

SEQ ID NO: 67 - protein, 10 aa, Homo sapiens
SSYTSSSTRV

SEQ ID NO: 68 - protein, 451 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 215 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 10 aa, Homo sapiens
GFTFSRYWMS

SEQ ID NO: 71 - protein, 17 aa, Homo sapiens
NIKQDGSEKYYVDSVKG

SEQ ID NO: 72 - protein, 12 aa, Homo sapiens
EGGWFGELAFDY

SEQ ID NO: 73 - protein, 12 aa, Homo sapiens
RASQRVSSSYLA

SEQ ID NO: 74 - protein, 7 aa, Homo sapiens
DASSRAT

SEQ ID NO: 75 - protein, 9 aa, Homo sapiens
QQYGSLPWT

SEQ ID NO: 76 - protein, 448 aa, Artificial Sequence
  Humanized sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 214 aa, Artificial Sequence
  Humanized sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 10 aa, Artificial Sequence
  Humanized sequence
GFTFSDSWIH

SEQ ID NO: 79 - protein, 18 aa, Artificial Sequence
  Humanized sequence
AWISPYGGSTYYADSVKG

SEQ ID NO: 80 - protein, 9 aa, Artificial Sequence
  Humanized sequence
RHWPGGFDY

SEQ ID NO: 81 - protein, 11 aa, Artificial Sequence
  Humanized sequence
RASQDVSTAVA

SEQ ID NO: 82 - protein, 7 aa, Artificial Sequence
  Humanized sequence
SASFLYS

SEQ ID NO: 83 - protein, 9 aa, Artificial Sequence
  Humanized sequence
QQYLYHPAT

SEQ ID NO: 84 - protein, 123 aa, Homo sapiens
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 85 - protein, 106 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 86 - protein, 5 aa, Homo sapiens
TYAIS

SEQ ID NO: 87 - protein, 17 aa, Homo sapiens
GIIPIFGKAHYAQKFQG

SEQ ID NO: 88 - protein, 14 aa, Homo sapiens
KFHFVSGSPFGMDV

SEQ ID NO: 89 - protein, 11 aa, Homo sapiens
RASQSVSSYLA

SEQ ID NO: 90 - protein, 7 aa, Homo sapiens
DASNRAT

SEQ ID NO: 91 - protein, 8 aa, Homo sapiens
QQRSNWPT

SEQ ID NO: 92 - protein, 439 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 214 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 5 aa, Homo sapiens
SYGMH

SEQ ID NO: 95 - protein, 17 aa, Homo sapiens
VIWYDGSNKYYADSVKG

SEQ ID NO: 96 - protein, 4 aa, Homo sapiens
NGDH

SEQ ID NO: 97 - protein, 11 aa, Homo sapiens
RASQSVSSNLA

SEQ ID NO: 98 - protein, 7 aa, Homo sapiens
GASTRAT

SEQ ID NO: 99 - protein, 9 aa, Homo sapiens
QQYNNWPRT

SEQ ID NO: 100 - protein, 468 aa, Homo sapiens
MEWSWVFLFFLSVTTGVHSEVQLVESGGGLVKPGGSLRLSCAASGFTYSNAWMSWVRQAPGKGLEWVGRIKSKTDGGTTDYAAPVQGRFTISRDDSKNTLYLQMNSLKTEDTAVYYCTIFGVVSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 234 aa, Homo sapiens
MSVPTQVLGLLLLWLTDARCDVQMTQSPSSLSASVGDRVTITCRASQSISDYLSWYQQRPGKAPNLLIYAASNLKTGVPSRFSGSGSGTDFTLTISTLQPEDFATYYCQQSYRSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 1404 bp, Homo sapiens
atggaatggagctgggtgttcctgttctttctgtccgtgaccacaggcgtgcattctgaagttcagctggtcgaaagcggaggaggtctggtgaaacccggtggctccctgaggctgagctgcgccgcctccggctttacttacagtaatgcctggatgtcctgggtcagacaggccccaggtaagggtctggagtgggtgggtaggattaagtctaaaactgatggcgggacaacagactatgccgccccagtgcaaggacggttcaccatttctagggacgactctaagaatacactgtatctgcagatgaacagcctcaaaacagaagacactgccgtttactactgtaccatctttggcgttgtctcctttgattattggggacagggtacactcgtgaccgtttcttccgcaagtacaaaggggccatcagtgtttccactggccccatcctctaagagcactagtggcggcacagccgccctgggatgtctggtgaaggactatttcccagagcctgtgaccgtcagctggaacagtggtgctctcacctcaggtgtgcacacattccccgctgtgctccaatccagtggcctctacagtctgagcagcgttgtgactgttcccagtagctcactgggcacccaaacctacatatgcaatgtgaaccataaacctagcaataccaaagtggacaagaaagtggaacctaagtcctgtgacaagactcatacctgtcctccttgtcctgccccagagctgctcggaggcccttccgtctttctcttcccaccaaagccaaaggataccctgatgatcagccggacacctgaggttacctgcgttgtggtcgacgtttcacacgaggatcctgaagtcaaattcaactggtacgttgatggagtcgaggtccacaacgccaaaaccaagcctcgcgaagaacaatacaatagcacatatagggtggtgtctgtgctcactgtcctgcaccaggactggctgaacggcaaggagtacaaatgcaaggttagtaacaaggccctgcccgcacccattgagaagactatcagtaaagctaagggccagcctcgcgagcctcaggtttacaccctgcctccctctagagaggaaatgacaaagaaccaggtgtctctcacctgcctggttaaaggattctatccatccgacattgctgtggaatgggaatccaacggacagcccgaaaacaactataagacaacaccacctgttctggattccgatggttccttctttctgtattccaaactcacagtggacaagagtcgctggcagcaaggtaacgtgttttcttgctccgtgatgcacgaagcactccacaatcactacactcagaagagtctcagcctctctccaggcaaa

SEQ ID NO: 103 - DNA, 702 bp, Homo sapiens
atgtctgtgcctacccaggtgctgggactgctgctgctgtggctgacagacgcccgctgtgatgttcagatgacacagtctccaagtagtctcagcgcaagcgttggcgacagagtgactatcacatgcagagcctctcagtctatctctgactatctgtcttggtaccagcagaggccaggcaaagctccaaacctcctgatctatgctgccagtaatctgaagacaggcgtgcctagtagattctccgggtccggtagtgggactgatttcaccctgacaatctccacactgcaacctgaggattttgctacctactattgtcagcaatcttatcgcagcccttggaccttcggacaggggactaaggttgagattaaacgcaccgtggcagcacccagcgtctttatctttcctccctccgacgagcagctcaagtccggaacagcatcagtcgtttgcctcctgaataacttttatccaagggaggccaaggtccagtggaaagtcgacaatgccctccaatctggtaactcccaggagtctgtgactgaacaagattctaaggacagtacctattcactcagctccaccctgaccctcagcaaagcagactacgaaaagcataaagtttacgcttgcgaagtgacccaccaaggcctgtcttctcctgtcacaaagagttttaatagaggggagtgt

SEQ ID NO: 104 - protein, 4 aa, Artificial Sequence
  Artificial linker
GFLG

SEQ ID NO: 105 - protein, 120 aa, Homo sapiens
QVQLVQSGAEVKKPGASVKVSCKASGYTFTGYYMHWVRQAPGQGLEWMGWINPNSGVSNYAQKFQGRFTMTRDTSISTLYMELSRLRSDDTAVYYCARANIAVAGAFDIWGQGTVVTVSS

SEQ ID NO: 106 - protein, 108 aa, Homo sapiens
DIQMTQSPSSLSASVGDRVTITCRASQGISNYLAWFQQKPGKAPKSLIYAASSLQRGVPSKFSGSGSGTDFTLIISSLQPEDFATYYCQQYISDPITFGQGTRLEIKR

SEQ ID NO: 107 - protein, 8 aa, Homo sapiens
GYTFTGYY

SEQ ID NO: 108 - protein, 8 aa, Homo sapiens
INPNSGVS

SEQ ID NO: 109 - protein, 11 aa, Homo sapiens
ANIAVAGAFDI

SEQ ID NO: 110 - protein, 6 aa, Homo sapiens
QGISNY

SEQ ID NO: 111 - protein, 7 aa, Homo sapiens
YAASSLQ

SEQ ID NO: 112 - protein, 9 aa, Homo sapiens
QQYISDPIT

SEQ ID NO: 113 - protein, 118 aa, Homo sapiens
EVQLVESGGGLIQPGGSLRLSCAASGFTVSSNYMSWVRQAPGKGLEWVSVIYSGGSTYYTDSVKGRFTISRDNSKNTLYLQMNSLRAEDTAVYYCARDKSGWNGFDYWGQGTLVTVSS

SEQ ID NO: 114 - protein, 108 aa, Homo sapiens
DVQMTQSPSSLSASVGDRVTITCRASQSVSTYLHWYQQKPGKAPKLLIYAASSLQGGLPSRFSGSGSGTDFTLTISSLQPEDFATYYCQQSSSTPLTFGGGTKVEITR

SEQ ID NO: 115 - protein, 8 aa, Homo sapiens
GFTVSSNY

SEQ ID NO: 116 - protein, 7 aa, Homo sapiens
IYSGGST

SEQ ID NO: 117 - protein, 10 aa, Homo sapiens
DKSGWNGFDY

SEQ ID NO: 118 - protein, 6 aa, Homo sapiens
QSVSTY

SEQ ID NO: 119 - protein, 7 aa, Homo sapiens
YAASSLQ

SEQ ID NO: 120 - protein, 9 aa, Homo sapiens
QQSSSTPLT